(19) United States
(12) Patent Application Publication
(10) Pub. No.: US 2010/0056637 A1
(43) Pub. Date: Mar. 4, 2010

(54) TREATMENT METHODS USING TRIARYL METHANE COMPOUNDS

(75) Inventors: Neil A. Castle, Cary, NC (US); Gregory C. Rigdon, Durham, NC (US); Douglas S. Krafte, Carrboro, NC (US); Jeffrey L. Krajewski, Raleigh, NC (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Icagen, Inc., Durham, NC (US)

(21) Appl. No.: 12/563,097

(22) Filed: Sep. 18, 2009

Related U.S. Application Data
(63) Continuation-in-part of application No. 12/233,937, filed on Sep. 19, 2008.
Application No. 12/233,937 is a continuation of application No. 11/642,416, filed on Dec. 20, 2006, now abandoned.
(60) Provisional application No. 61/098,589, filed on Sep. 19, 2008.
(60) Provisional application No. 60/752,935, filed on Dec. 20, 2005.

(51) Int. Cl. A61K 31/165; A61P 11/06

(52) U.S. Cl. 514/617

(57) ABSTRACT

The present invention provides methods of treating or preventing asthma or an inflammatory disease. In one embodiment, the invention provides compounds and formulations for the treatment of asthma or an inflammatory disease.

### CROSS-REFERENCES TO RELATED APPLICATIONS

This application claims priority benefit of U.S. Patent Application Ser. No. 61/098,589, filed Sep. 19, 2008; this application is also a continuation-in-part of U.S. patent application Ser. No. 12/233,937 filed Sep. 19, 2008, which was a continuation application of U.S. patent application Ser. No. 11/642,416 filed on Dec. 20, 2006, which claimed priority benefit of U.S. Patent Application Ser. No. 60/752,935, filed on Dec. 20, 2005 the disclosures of each of which are incorporated herein by reference in their entireties for all purposes.

### STATEMENT AS TO RIGHTS TO INVENTIONS MADE UNDER FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

Asthma affects up to 10% of the world's population at some point during their lives. Despite the availability of a wide variety of pharmacological interventions for asthma, the disease is still inadequately controlled in many patients. Of those affected, 33% use a rescue inhaler daily and 71% would take a new medication if it were available. Airway responses result from the orchestrated activation of a variety of immunologically active cells. The cellular processes underlying many of these events require a sustained elevation of intracellular calcium. Preventing or restricting cellular calcium entry would therefore be predicted to be of benefit. Maintenance of elevated intracellular calcium is typically aided by potassium channel activation which hyperpolarizes the cell and sustains the driving force for calcium entry. The calcium activated potassium channel called KCa3.1 plays this role in many inflammatory cell types. Currently, the specific roles that individual inflammatory cell types play in asthma remain to be fully resolved. Affecting migration or infiltration of cells such as mast cells may be particularly beneficial in chronic diseases such as asthma. Since certain inflammatory cells already reside in lung tissue it is possible that therapeutic benefit from inhibition of these processes may take several days or longer since inhibition of new cell recruitment and infiltration could be the primary cause of therapeutic effect.

One potential cell type that may be targeted for asthma therapy is mast cells. It is known that mast cells are recruited to and activated at sites of inflammation and fibrosis. These cells invade diseased tissues such as airway smooth muscle (ASM) in asthma patients. Mast cells express the K⁺ channel protein KCa3.1 on their surface, which is involved with the release of mediator proteins in vivo. Additionally, it was recently shown that KCa3.1 was involved in ligand interactions important for chemotaxis (Cruse et al., Thorax. 2006 October; 61(10):880-5).

These potassium channels are good targets for drug candidates because of their restricted expression. They are predominantly expressed in blood and epithelial cells. The specific expression pattern of these channels suggests that specific K⁺-channel inhibitors may have fewer side-effects.

### BRIEF SUMMARY OF THE INVENTION

The present invention is particularly useful in treating or preventing asthma. Thus the present invention provides a method of treating or preventing asthma. The method includes administering to a subject suffering from asthma a therapeutically effective amount of a compound having the structure according to Formula (I), Formula (II), Formula (III), Formula (IV), or Formula (V). Additionally, the present invention provides compositions and formulation useful in the treatment of asthma.

Triphenylacetamide-based K⁺-channel blockers are promising candidates for the treatment of sickle cell disease (SCD) as discussed in U.S. Pat. No. 6,288,122 which is herein incorporated by reference. In addition, studies indicate that triphenylacetamide-based inhibitors are potential candidate drugs for the treatment of inflammatory conditions. In vitro studies show that a triphenylacetamide-based inhibitor, compound 3 in Table 1, which has a long half-life in blood, including human blood, inhibits K⁺ channels with a high selectivity for the IK1 channel. In vivo studies of 2,2-bis(4-fluorophenyl)-2-phenylacetamide, also referred to herein as “Senicapoc”, “or Formula (V), demonstrate that it has a long half in blood, including human blood (Ataga et al., Pharmacotherapy, 26(11):1557-64 (2006)).

Thus, in the first aspect the invention provides a method of treating or preventing asthma. The method includes administering to a subject suffering from asthma, or at risk of developing asthma, a therapeutically effective amount of a compound having the structure according to (I), Formula (II), Formula (III), Formula (IV), or Formula (V). In a preferred embodiment, the compound has a structure according to Formula (V) In another exemplary embodiment, the method involves treating or preventing asthma by administering a compound of the invention to a mammal not otherwise in need of treatment with the compounds of the invention. In certain embodiments, the invention provides methods of preventing the migration of mast cells or the degranulation of mast cells. In yet other embodiments, the invention provides methods of reducing the action of mast cells during an asthma attack or reducing the action of mast cells in the development of asthma. When selecting the patient population for treatment of asthma with the compounds of the invention, there exists a proviso that the patient does not suffer from sickle cell disease.

In a second aspect, the compounds for use according to the invention are also useful in treating and preventing an inflammatory process. Accordingly, the present invention also provides a method for treating or preventing an inflammatory process, said method comprising administering to a subject suffering from said inflammatory process a therapeutically effective amount of a compound having the structure according to Formula (I), Formula (II), Formula (III), Formula (IV), or Formula (V). When selecting the patient population for treatment of inflammatory disorders with the compounds of the invention, there exists a proviso that the patient does not suffer from sickle cell disease.

The compounds for use according to the invention in any aspect are according to Formula (I), Formula (II), Formula (III), Formula (IV), or Formula (V) are next described:

### Formula 1:

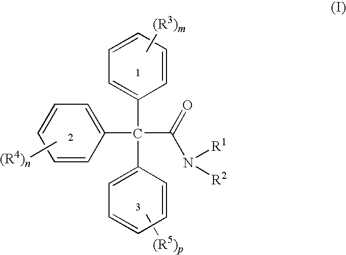

wherein R³, R⁴, and R⁵ are independently selected from F and CF₃, wherein m, n and p are independently selected from 0, 1, 2, and 3, wherein at least one of m, n and p is not 0, and wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl. In one embodiment, at least one of R¹ and R² is an alkyl selected from methyl, ethyl, n-propyl, isopropyl, n-butyl, and isobutyl. In a particular embodiment at least one of R¹ and R² is H. In another particular embodiment, both of R¹ and R² are H.

In one embodiment, when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent. In another exemplary embodiment, when p is 0, and m is 1 and n is 1, the fluoro substituent at ring 1 is para to the acetamide substituent, and the substituent at ring 2 is located at a position selected from ortho to the acetamide substituent and para to the acetamide substituent.

Controlling inflammatory processes via altering cellular ionic fluxes of cells affected by a disease is a powerful therapeutic approach. Moreover, basic understanding of the role of cellular ionic fluxes in both disease processes and normal physiology promises to provide new therapeutic modalities, regimens and agents. Compounds that alter cellular ion fluxes, particularly those that inhibit potassium flux, are highly desirable as both drugs and as probes for elucidating the basic mechanisms underlying these ion fluxes. Similarly, methods utilizing these compounds in basic research and in therapeutic applications are valuable tools in the arsenal of both the researcher and clinician. Therefore such compounds and methods are also an object of the present invention.

Thus, in third aspect, the present invention provides a method of inhibiting potassium flux of a cell. The method includes contacting a cell with an amount of a compound according to Formula (I), Formula (II), Formula (III), Formula (IV), or Formula (V) effective to inhibit the potassium flux.

In a fourth aspect the invention provides a method of preventing or retarding autoreactive T-cell growth. An important therapeutic pathway for the treatment of asthma and also for an inflammatory process is preventing or retarding autoreactive T-cell growth. This growth retardation can be accomplished by manipulating the cellular ion fluxes of the T-cells. Thus, in another aspect, the invention provides a method for preventing or retarding autoreactive T-cell growth. The method includes contacting a T-cell with an amount of a compound according to Formula (I), Formula (II), Formula (III), Formula (IV), or Formula (V) effective for preventing or retarding autoreactive T-cell growth.

In a fifth aspect, the invention provides pharmaceutical formulations and compositions useful for the treatment of asthma, comprising a compound having a structure as given in Formula (I), Formula (II), Formula (III), Formula (IV), or Formula (V).

In a sixth aspect, the invention provides pharmaceutical formulations and compositions useful for the treatment of an inflammatory process or disease, comprising a compound having a structure as given in Formula (I), Formula (II), Formula (III), Formula (IV), or Formula (V).

These and other objects and advantages of the present invention will be apparent from the detailed description and examples that follow.

### BRIEF DESCRIPTION OF THE DRAWINGS

FIG. 1. Senicapoc inhibits migratory response in isolated human lung mast cells. Results of HLMC chemotaxis assays performed, using a method as given in Cruse et al. 2006, on isolated human mast cells in the presence and absence of Senicapoc.

FIG. 2. Senicapoc inhibits KCa3.1 currents in CHO cells stably expressing KCa3.1 and in isolated human lung mast cells. Stimulated cells were subject to whole patch clamp experiments with currents detected in the presence of varying concentrations of Senicapoc (left panel). Alternatively, the voltage applied in the patch clamp experiments was varied and current measured at varying concentrations of Senicapoc (right panel).

FIG. 3. Effect of aerosol administration of Senicapoc using a sheep asthma model similar to as described in Abraham (Pulm Pharmacol Ther. 2008; 21(5):743-54). The fold increase in baseline lung resistance (R_{L}) upon A. suum antigen challenge was determined at various time points after aerosol administration of 30 mg/kg Senicapoc or vehicle control (left panel). Airway hyper-reactivity was measured at 24 hours post allergen challenge based on increasing doses of carbachol.

FIG. 4. Effect of intravenous administration of Senicapoc using a sheep asthma model similar to as described in Abraham (Pulm Pharmacol Ther. 2008; 21(5):743-54). The fold increase in baseline lung resistance (R_{L}) upon A. suum antigen challenge was determined at various time points after intravenous administration of 3 mg/kg Senicapoc, 10 mg/kg Senicapoc, or vehicle control (left panel). Airway hyper-reactivity was measured at 24 hours post allergen challenge based on increasing doses of carbachol.

FIG. 5. Effect of oral administration of Senicapoc using a sheep asthma model similar to as described in Abraham (Pulm Pharmacol Ther. 2008; 21(5):743-54). The fold increase in baseline lung resistance (R_{L}) upon A. suum antigen challenge was determined at various time points after oral administration of 10 mg/kg Senicapoc, 30 mg/kg Senicapoc, or vehicle control (left panel). Airway hyper-reactivity was measured at 24 hours post allergen challenge based on increasing doses of carbachol.

FIG. 6. Pharmacokinetics of Senicapoc in sheep plasma following 30 mg/kg po b.i.d. Plasma was collected from sheep at the indicated times after oral administration of Senicapoc, A. suum antigen challenge, and carbachol administration.

FIG. 7. Pharmacokinetics of Senicapoc in man following maintenance doses (MD) of 15, 20, 30, and 40 mg/day. Plasma was collected from normal, healthy volunteers at the indicated times after oral administration of Senicapoc. LD indicates loading dose on day 1 followed by a maintenance dose (MD) given once per day for the duration of the study. The dashed line indicates the average efficacious level of Senicapoc in the sheep model of asthma following oral administration.

FIG. 8. Process flow diagram of a manufacturing process for tablets of Senicapoc used for oral administration in humans.

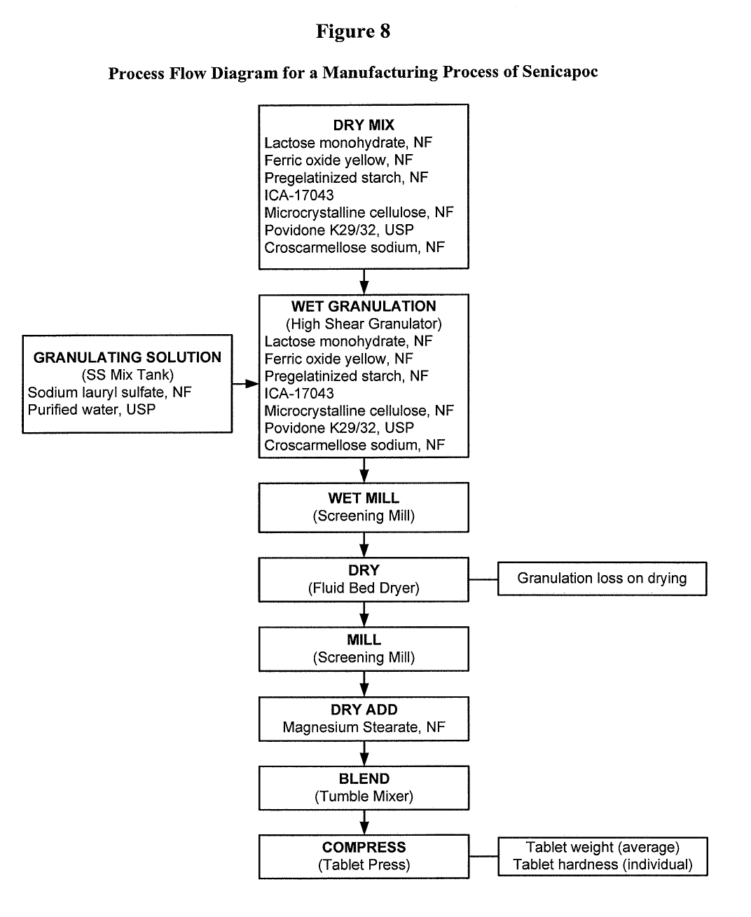

FIG. 9. FIG. 9A study design. A loading dose of 80 mg BID for three days of Senicapoc was followed by a daily maintenance dose of 40 mg. FIG. 9B. Change in FEV₁ over time past allergen challenge (AC) in human subjects. The late allergen response (LAR) is the decline in FEV1 4 to 10 hours after patients were challenged with an inhaled allergen. The magnitude of the decline in LAR is quantified as the area bounded by the baseline FEV₁ (0%), and the actual FEV₁ measurements between hours 4 and 10. Subjects randomized to 13 days of treatment with placebo had no change in the LAR (left panel). By contrast, patients randomized to receive Senicapoc, had less of an LAR. By analysis of co-variance, the difference in LAR at Day 13 between the two treatment groups was 29%. In addition, the fraction of exhaled nitric oxide was decreased after 13 days of treatment in the senicapoc group, compared to no change in the placebo group.

### DETAILED DESCRIPTION OF THE INVENTION

### Abbreviations and Definitions

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Fluoroalkyl” refers to a subclass of “substituted alkyl” encompassing alkyl or substituted alkyl groups that are either partially fluorinated or per-fluorinated. The fluorine substitution can be the only substitution of the alkyl moiety or it can be in substantially any combination with any other substituent or group of substituents.

In some embodiments, the invention provides methods of treating or preventing “asthmatic bronchitis”, “exercise-induced asthma” (EIA), or “exercise induced bronchial spasm” as used herein refer to a chronic condition characterized by at least one symptom such as intermittent airway constriction, bronchial spasms, excessive airway mucus, hyperresponsiveness to allergen stimuli, eosinophilia, constriction of airway smooth muscle, edema and hypersecretion of mucous leading to increased work in breathing, dyspnea, hypoxemia, hypercapnia. and the like.

In some embodiments, the invention provides a method of treating asthma or an asthmatic attack. An asthma attack may be caused by one of many stimuli including environmental, infectious, and internal stimuli. Asthma may be characterized by at least one symptom such as intermittent airway constriction, bronchial spasms, excessive airway mucus, hyperresponsiveness to allergen stimuli, eosinophilia, constriction of airway smooth muscle, edema and hypersecretion of mucous leading to increased work in breathing, dyspnea, hypoxemia, hypercapnia. and the like. Generally, an asthma attack is the result of cellular histamine release after exposure to a stimulus or antigen. It is thought that mast cells are present at the surface of lung tissue and in related areas, and are further recruited to the lung surface and related tissues upon continued stimulation; and these cells are thought to be responsible for the histamine release, by a process involving degranulation of the mast cells. Chronic over-recruitment of mast cells to the lungs can damage the tissues contributing to chronic asthma problems. In certain cases, asthma may be stimulated by allergens, including ragweed, dust mites, pollutants, smoke, respiratory infections, pollens, pet dander, mold, mildew, as well as cold air, exercise, stress, anxiety, or other stimuli.

### Compounds

In its various aspects, the present invention utilizes a compound having a structure according to Formula (I):

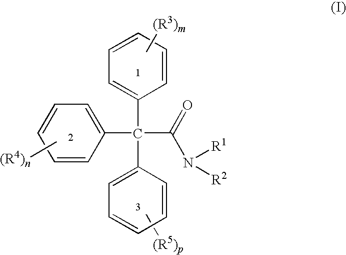

wherein R³, R⁴, and R⁵ are independently selected from F and CF₃, wherein m, n and p are independently selected from 0, 1, 2, and 3, wherein at least one of m, n and p is not 0, and wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, an ether, an ester, a hydroxyl, and the like. In one embodiment, at least one of R¹ and R² is an alkyl selected from methyl, ethyl, n-propyl, isopropyl, n-butyl, and isobutyl. In a particular embodiment at least one of R¹ and R² is H. In another particular embodiment, both of R¹ and R² are H.

In another embodiment, at least one of R³, R⁴, or R⁵ is CF₃. In another embodiment wherein m, n and p are independently selected from 0, 1 and 2, at least one of R³ is CF₃, or at least one of R⁴ is CF₃, or at least one of R⁵ is CF₃. In another embodiment, wherein m, n and p are independently selected from 0, 1 and 2 wherein at least one of m, n and p is not 0, R³, R⁴, and R⁵ are independently selected from CF₃. In another embodiment, wherein m, n and p are independently selected from 0 and 1 wherein at least one of m, n and p is not 0, R⁴, and R⁵ are independently selected from CF₃. In another embodiment of the above, R¹ and R² are both H or not both H. In another embodiment at least one of R¹ and R² is substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, an ether, an ester, a hydroxyl. In a further embodiment of the above one of R¹ and R² is H.

In one embodiment, when in, n and p are all 1, the fluoro substituents at ring I and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent. In another exemplary embodiment, when p is 0, and m is 1 and n is 1, the fluoro substituent at ring 1 is para to the acetamide substituent, and the substituent at ring 2 is located at a position selected from ortho to the acetamide substituent and para to the acetamide substituent.

In yet other embodiments of the invention, one or more of R¹ and R² may alternatively be selected from the group consisting of an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, an ether, and ester, a halogen, a cyano, an azide, a hydroxyl, and the like. In particular embodiments, R³, R⁴, and R⁵ may be methyl, ethyl, n-propyl, isopropyl, n-butyl, isobutyl, a C₅ alkyl, a C₆ alkyl, CN, N₃, a C₁ to C₆ alkyl ester, F, Cl, Br, I, and the like.

In another embodiment, the compounds utilized in the present invention have a structure according to Formula (II):

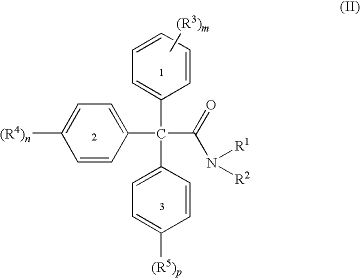

wherein R³, R⁴, and R⁵ are independently selected from F and CF₃, wherein m, n and p are independently selected from 0, 1, 2, and 3, wherein at least one of m, n and p is not 0, and wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, an ether, an ester, a hydroxyl, and the like. In one embodiment, at least one of R¹ and R² is an alkyl selected from methyl, ethyl, n-propyl, isopropyl, n-butyl, and isobutyl. In a particular embodiment at least one of R¹ and R² is H. In another particular embodiment, both of R¹ and R² are H.

In another embodiment, the compounds of the invention have a structure according to Formula III:

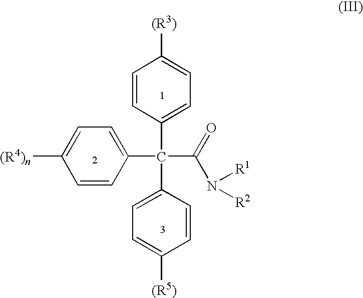

wherein R³, R⁴, and R⁵ are independently selected from F and CF₃, wherein n is 0, 1, 2, or 3, and wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, an ether, an ester, a hydroxyl, and the like. In one embodiment, at least one of R¹ and R² is an alkyl selected from methyl, ethyl, n-propyl, isopropyl, n-butyl, and isobutyl. In a particular embodiment at least one of R¹ and R² is H. In another particular embodiment, both of R¹ and R² are H.

In another embodiment, the compounds of the invention have a structure according to Formula IV:

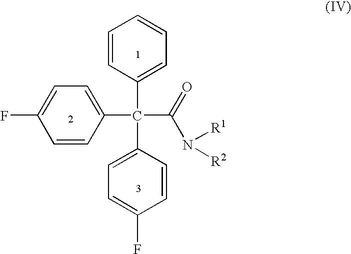

wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, an ether, an ester, a hydroxyl, and the like. In one embodiment, at least one of R¹ and R² is an alkyl selected from methyl, ethyl, n-propyl, isopropyl, n-butyl, and isobutyl. In a particular embodiment at least one of R' and R² is H. In another particular embodiment, both of R¹ and R² are H.

In a particular embodiment, the compounds to be utilized according to the invention have a structure according to Formula V:

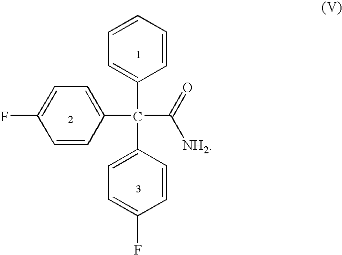

In additional embodiments, the present invention utilizes a compound having a structure according to Formula (Ia):

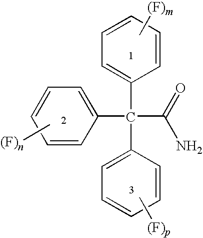

wherein m, n and p are independently selected from 0 and 1 and at least one of m, n and p is 1. In an exemplary embodiment of the same, when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent. In another exemplary embodiment, when p is 0, and m is 1 and n is 1, the fluoro substituent at ring 1 is para to the acetamide substituent, and the substituent at ring 2 is located at a position selected from ortho to the acetamide substituent and para to the acetamide substituent.

In another exemplary embodiment, the compounds utilized in the present invention have a structure according to Formula (IIa):

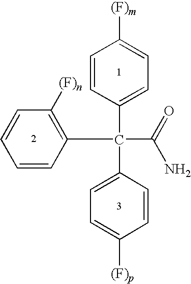

wherein m, n and p are independently selected from 0 and 1, and at least one of m, n and p is 1.

Compounds according to this structure are displayed in Table 1.

In another exemplary embodiment, the compounds of the invention have a structure according to Formula IIIa:

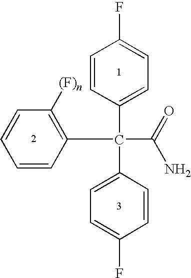

wherein n is either 0 or 1.

Compounds that are structurally closely related to compounds of the invention are also displayed in Table 1. The compounds which are structurally related to the compounds of the invention serve as a “baseline” for assessing the advantages and unexpected properties and benefits of the fluorinated compounds of the invention.

TABLE 1

| | |
|---|---|
| | (1) |
| 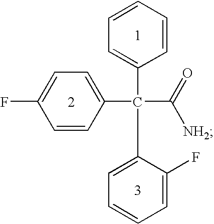 | |
| | (2) |
| 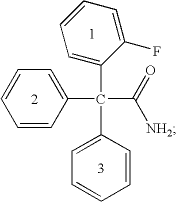 | |
| | (3) |
| 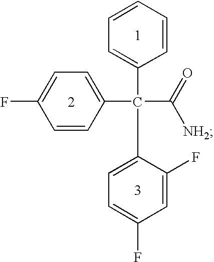 | |
| | (4) |
| 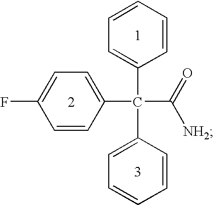 | |
| | (5) |
| 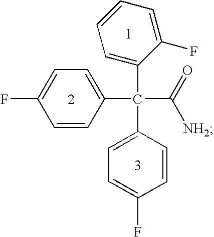 | |
| | (6) |
| 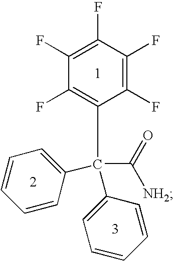 | |
| | (7) |
| 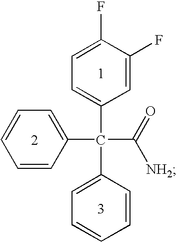 | |
| | (8) |
| 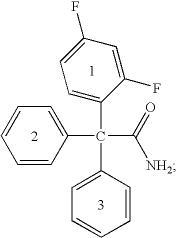 | |
| | (9) |
| 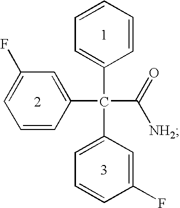 | |

| | |
|---|---|
| | (10) |
| 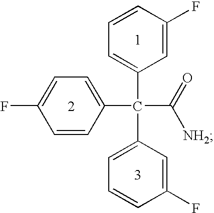 | |
| | (11) |
| 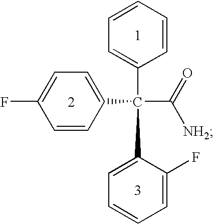 | |
| | (12) |
| 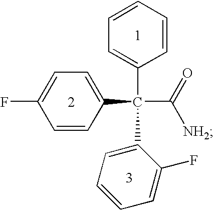 | |
| | (13) |
| 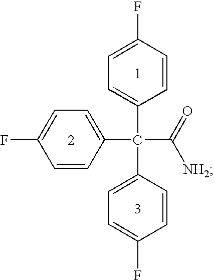 | |
| | (14) |
| 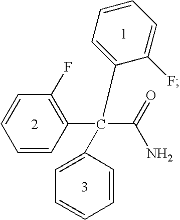 | |
| | (15) |
| 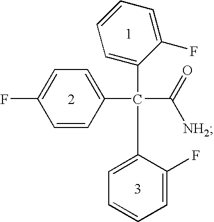 | |
| | (16) |
| 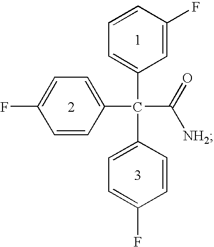 | |
| | (17) |
| 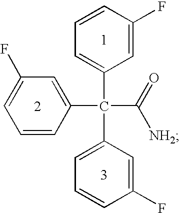 | |
| | (18) |
| 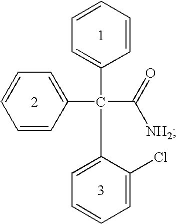 | |
| | (19) |
| 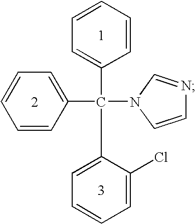 | |
| | (20) |
| 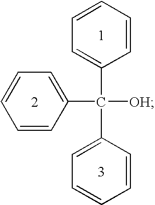 | |
| | (21) |
| 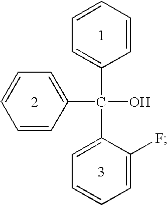 | |
| | (22) |
| 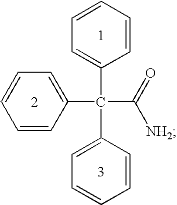 | |
| | (23) |
| 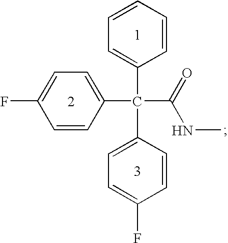 | |
| | (24) |
| 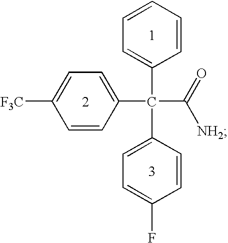 | |
| | (25) |
| 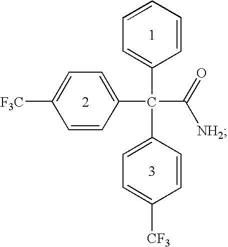 | |
| | (26) |
| 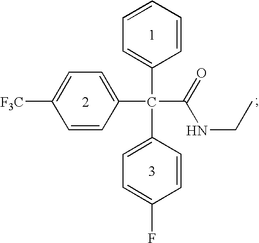 | |
| | (27) |
| 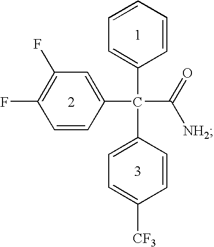 | |
| | (28) |
| 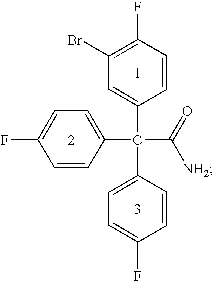 | |
| | (29) |
| 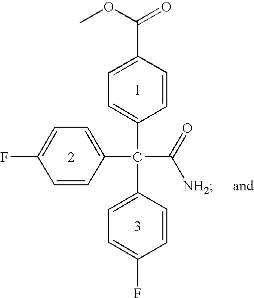 | |
| | (30) |
| 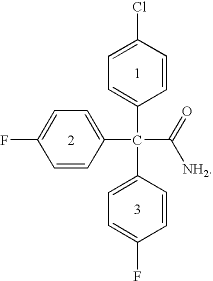 | |

In a particular aspect, the invention provides a method of treating asthma with a compound having a structure according to Formula V.

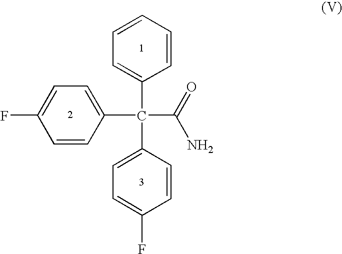

In certain aspects, the invention provides formulations useful for the treatment of asthma comprising a compound according to Formula V. As used herein, “2,2-bis(4-fluorophenyl)-2-phenylacetamide”, “Senicapoc” all refer to a compound having the structure according to Formula V.

Advantageously, Senicapoc displays favorable pharmacokinetic properties, as seen in a number of recent clinical studies. Notably, 68 patients were administered an oral dose of this compound in three clinical studies. Mean elimination half-life of the compound across the three studies was 12-17 days, as reported in Ataga et al., Pharmacotherapy (2006). Similarly, phase I clinical studies showed no drug-related serious adverse events. In dose-escalation studies of the compound (Ataga et al., Pharmacotherapy (2006)), the terminal half-life of the compound was 370 hours, 219 hours, and 297 hours in patients with sickle cell disease administered 50, 100, and 150 mg of drug in a single dose. Mean Cₘₐₓ values for these patient cohorts were 59.1, 108.7, and 109.1 ng/ml respectively. The mean total systemic dose of the compound for the cohorts were 11,826, 19,696, and 30,675 (ng·hr/ml) respectively. Finally, the reported oral clearance rates were 4.75, 6.1, and 5.8 L/hr respectively. Given these long half-lives and favorable pharmacokinetic properties, the compound is particularly well suited for administration in vivo.

### Compound Synthesis

The compounds of the invention can be prepared by techniques that are standard in the art of organic synthesis. Appropriate starting materials and reagents can be obtained commercially or they can be prepared by standard organic chemistry techniques. Exemplary processes are illustrated by the specific examples. An exemplary synthetic route is provided in Scheme 1.

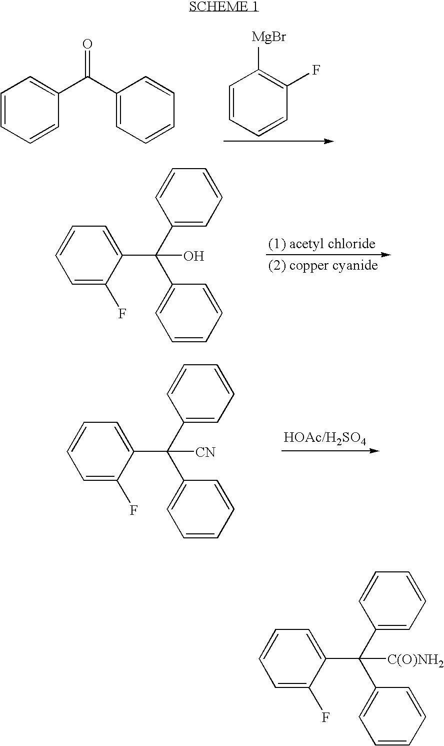

In Scheme 1, the synthesis of a fluorine-substituted triphenylacetamide proceeds from the corresponding fluorine-substituted triphenylmethanol that is prepared from a fluorine-substituted benzophenone and a reagent that adds a phenyl or fluorine-substituted phenyl moiety to the benzophenone ketone. The fluorine-substituted triphenylmethanol is subsequently converted to the corresponding fluorine-substituted triphenylacetonitrile by exposing the alcohol to acetyl chloride followed by copper cyanide. The acetamide can be formed by reacting the intermediate nitrile with a mixture of sulfuric and glacial acetic acids. Other synthetic routes leading to fluorine-substituted triphenylmethane species, particularly acetamides, are within the abilities of those skilled in the art.

### Compound Stability

For compounds to act as pharmaceutically useful KCa3.1 channel inhibitors, candidate compounds must demonstrate both acceptable bioavailability and stability in vivo. Subjects undergoing treatment should be regularly dosed with the compound of the invention during the dosage loading period. The dosage loading period can be one day, or two days or three days, or four days or up to a week or two, depending upon the compound used. For chronic inflammatory diseases or asthma, the dosage loading period can then be followed by a dosage maintenance period, and it is best to dose subjects in a consistent manner with a compound or compounds of the invention at regular intervals during the dosage maintenance period to help reduce chronic effects of the disease, such as prolonged over-recruitment of mast cells to the affected organs (e.g., the lungs). Compounds having increased in vivo residence times and increased bioavailability allow for a simplified dosage regimen (i.e. fewer doses/day and/or less medication). Moreover, reducing the amount of compound administered carries with it the promise of reducing side effects resulting from the medication and/or its metabolites. Thus, it is highly desirable to provide KCa3.1 channel inhibitors which demonstrate good bioavailability and enhanced in vivo stability.

### Compound Activity

To develop pharmaceutically useful KCa3.1 channel inhibitors, candidate compounds must demonstrate acceptable activity towards the target channel. Compounds are judged to be sufficiently potent if they have an IC₅₀ towards the KCa3.1 channel of no more than 100-500 nM. For example, an IC₅₀ of a compound of this invention, as determined in vitro or in vivo in a mast cell migration assay; or in a mast cell degranulation assay; which is <5 nM, or <10 nM, <50 nM, or <100 nM, or <200 nM or <300 nM, or <400 nM, or <500 nM would be of value for use in this invention. Chemotaxis or migration assays suitable for use in conjunction with the present invention are well known in the art and include, without limitation, those reviewed in Zigmond et al. (Curr Protoc Cell Biol. 2001 May; Chapter 12:Unit 12.1) and Entschladen et al. (Exp Cell Res. 2005 Jul. 15; 307(2):418-26). Similarly, mast cell degranulation assays are well known in the art and include, without limitation, flow cytometry assays (Demo et al., Cytometry 1999 Aug. 1; 36(4):340-8.), single cell staining assays (Windmiller and Backers, J. Biol. Chem., Vol. 278, Issue 14, 11874-11878, Apr. 4, 2003), assays measuring hexosaminidase assay (Choi et al., The Journal of Immunology, Vol 151, Issue 10 5586-5595), and the like.

The activity of the compounds of the invention towards ion channels can be assayed utilizing methods known in the art. For example, see, Brugnara et al., J. Biol. Chem., 268(12): 8760-8768 (1993). Utilizing the methods described in this reference, both the percent inhibition of the Gardos channel and the IC₅₀ of the compounds of the invention can be assayed.

Other methods for assaying the activity of ion channels and the activity of other agents that affect the ion channels are known in the art. The selection of appropriate assay methods is well within the capabilities of those of skill in the art. See, for example, Hille, B., Ionic Channels Of Excitable Membranes, Sinaner Associates, Inc., Sunderland, Mass. (1992).

### Compound Selectivity

For compounds to act as pharmaceutically useful KCa3.1 channel inhibitors, candidate compounds must demonstrate acceptable selectivity towards the target channel. Compounds having a selectivity towards the Gardos channel or KCa3.1 vs other potassium channels of at least 30 fold are judged to be sufficiently selective. The selectivity ranges which are useful for the compounds of the invention can also be higher than this (more selective), such as >30 fold, or >40 fold, or >50 fold, or >100 fold, depending upon the compound used and what selectivity is required for use in a subject to achieve an adequate selectivity.

The selectivity of a particular compound for the KCa3.1 channel relative to another potassium ion channel is conveniently determined as a ratio of two compound binding-related quantities (e.g., IC₅₀). In an exemplary embodiment, the selectivity is determined using the activities determined as discussed above, however, other methods for assaying the activity of ion channels and the activity of agents that affect the ion channels are known in the art. The selection of appropriate assay methods is well within the capabilities of those of skill in the art. See, for example, Hille, B., Ionic Channels Of Excitable Membranes, Sinaner Associates, Inc., Sunderland, Mass. (1992).

In one embodiment, the compounds of the invention are potent, selective and stable inhibitors of potassium flux, such as that mediated by the KCa3.1 channel.

While not wishing to be bound by any particular theory of operation, it is presently believed that certain structural features of the compounds of the invention (i.e. replacement of hydrogen with fluorine) are presently implicated in the stability, selectivity and potency of these compounds. Thus, in an exemplary embodiment, the inhibitors of the invention include an aryl moiety, wherein at least one hydrogen atom of the aryl moiety is replaced by a radical comprising a fluorine atom. In this embodiment, the invention encompasses fluorinated derivatives of compounds that inhibit potassium ion flux, particularly those having KCa3.1 channel inhibitory activity (e.g., antimycotic agents, e.g., miconazole, econazole, butoconazole, oxiconazole and sulconazole). Other agents that have potassium ion channel inhibitory activity, and particularly KCa3.1 channel inhibitory activity, and possess at least one aryl moiety bearing at least one fluorine atom are within the scope of the present invention.

In some cases, the presence of at least two fluorine radicals in the structure of a compound of the invention is beneficial for stabilizing the metabolic reactivity and the stability of the compound in a mammal, such as in a human. The fluorine radicals can be located on one aryl ring or on different aryl rings of the compound. In some cases, the half life of difluorinated and trifluorinated compounds of the invention can be extended in the blood of a mammal, such as in a human, such that the average half life is found to be greater than 10 hours; or greater than 20 hours; or greater than 40 hours; or is greater than 100 hours in mammalian blood, as determined in vivo or in vitro, or ex vivo, and the extended half life is additionally beneficial for use in the treatment of asthma, inflammatory diseases, MS, or pulmonary hypertension.

In a particular embodiment, one or more of the aryl moieties is a phenyl group or a substituted phenyl group. In another exemplary embodiment, the three aryl moieties found in compounds of the invention together constituent a mono-, di-, or tri-substituted triphenylmethyl group.

The compound(s) of the invention can be administered per se or in the form of a pharmaceutical composition wherein the active compound(s) is in admixture with one or more pharmaceutically acceptable carriers, excipients or diluents. Thus, in addition to compounds that affect cellular ion fluxes (e.g., KCa3.1 channel inhibiting activity), the present invention also provides pharmaceutical formulations that contain the compounds of the invention.

### Pharmaceutical Formulations

In a second aspect, the invention provides a pharmaceutical formulation comprising a compound of the invention according to Formula (I) admixed with a pharmaceutically acceptable excipient. In an exemplary embodiment, the compounds are those according to Formula (II), Formula (III), Formula (IV), or Formula (V).

The compounds described herein, or pharmaceutically acceptable addition salts or hydrates thereof, can be formulated so as to be delivered to a patient using a wide variety of routes or modes of administration. Suitable routes of administration include, but are not limited to, inhalation, transdermal, oral, ocular, rectal, transmucosal, intestinal and parenteral administration, including intramuscular, subcutaneous and intravenous injections.

The compounds described herein, or pharmaceutically acceptable salts and/or hydrates thereof, may be administered singly, in combination with other compounds of the invention, and/or in cocktails combined with other therapeutic agents. The choice of therapeutic agents that can be co-administered with the compounds of the invention will depend, in part, on the condition being treated.

For example, when administered to patients suffering from asthma or an inflammatory process, or multiple sclerosis, the compounds of the invention can be administered in cocktails containing agents used to treat the pain, infection and other symptoms and side effects commonly associated with an inflammatory process. Such agents include, e.g. analgesics, antibiotics, etc. The compounds can also be administered in cocktails containing other agents that are commonly used in treating inflammatory process, including butyrate and butyrate derivatives (Perrin et al., N. Engl. J. Med. 328(2): 81-86 (1993)); hydroxyurea (Charache et al., N. Engl. J. Med. 323(20): 1317-1322 (1995)); erythropoietin (Goldberg et al, N. Engl. J. Med. 323(6): 366-372 (1990)); and dietary salts such as magnesium (De Franceschi et al., Blood 88(648a): 2580 (1996)).

Pharmaceutical compositions for use in accordance with the present invention can be formulated in a conventional manner using one or more physiologically acceptable carriers comprising excipients and auxiliaries, which facilitate processing of the active compounds into preparations which can be used pharmaceutically. Proper formulation is dependent upon the route of administration chosen.

For injection, the agents of the invention can be formulated in aqueous solutions, preferably in physiologically compatible buffers such as Hanks's solution, Ringer's solution, or physiological saline buffer. In an exemplary embodiment, the formulation includes water and an alcohol and/or glycol. Other useful components of this formulation include, for example, surfactant, emulsifiers and materials such as ethoxylated oils. An exemplary formulation includes a compound of the invention, poly(ethyleneglycol) 400, ethanol and water in a 1:1:1 ratio. Another exemplary formulation includes a compound of the invention, water, poly(ethyleneglycol) 400 and Cremophor-EL.

For transmucosal administration (e.g., buccal, rectal, nasal, ocular, etc.), penetrants appropriate to the barrier to be permeated are used in the formulation. Such penetrants are generally known in the art.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be combined with a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents may be added, such as the cross-linked polyvinyl pyrrolidone, agar, or alginic acid or a salt thereof such as sodium alginate.

Pharmaceutical formulations for parenteral administration include aqueous solutions of the active compounds in water-soluble form, such as those described above for intravenous administration. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may include suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

### Effective Dosages

Pharmaceutical compositions suitable for use with the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to reduce the occurrence of multiple sclerosis and/or impair the formation of autoreactive T-cells, such compositions will contain an amount of active ingredient effective to achieve this result. In a second example, when the compositions of the invention are administered to treat or reduce the occurrence of asthma or reduce the migration of mast cells or the degranulation of mast cells, such compositions will contain an amount of active ingredient effective to achieve this result. Determination of an effective amount is well within the capabilities of those skilled in the art, especially in light of the detailed disclosure herein.

For any compound described herein, the therapeutically effective amount can be initially determined from cell culture assays. Target plasma concentrations will be those concentrations of active compound(s) that are capable of inducing inhibition of the IK1 channel. In exemplary embodiments, the KCa3.1 channel activity is at least 25% inhibited. Target plasma concentrations of active compound(s) that are capable of inducing at least about 50%, 75%, or even 90% or higher inhibition of the KCa3.1 channel potassium flux are presently preferred. The percentage of inhibition of the KCa3.1 channel in the patient can be monitored to assess the appropriateness of the plasma drug concentration achieved, and the dosage can be adjusted upwards or downwards to achieve the desired percentage of inhibition.

In certain embodiments, the therapeutically effective dose can be determined based on the inhibition of an allergen response upon antigen challenge, for example by measuring the increase in lung resistance upon allergen challenge, carbachol-induced airway hyper-reactivity after allergen challenge, or mast cell degranulation after allergen challenge. For example, a therapeutically effective dose in some embodiments may be the amount capable of reducing lung resistance by 10% after antigen or allergen challenge. In other embodiments, the reduction in lung resistance resulting from administration of a therapeutically effective amount or dose of a compound of the invention may be from about 20% to about 100%, or from about 40% to about 70%. In other embodiments, the inhibition may be at least about 20%, or about 30%, 40%, 50%, 60%, 70%, 80%, 90%, 95%, or more inhibition of lung resistance. In yet other embodiments, the lung resistance resulting from a therapeutically effective dose may be about 1-fold, 2-fold, 3-fold, 4-fold, 5-fold, or more reduced as compared to administration of a vector or negative control after the allergen challenge. In other embodiments, a therapeutically effective dose may be the amount required to increase carbachol-induced airway hyper-reactivity after allergen challenge by at least 10%. In other embodiments, an effective dose may raise the carbachol-induced airway hyper-reactivity in a patient by at least about 20%, or 30%, 40%, 50%, 60%, 70%, 80%, 90%, 100% or more, as compared to administration of a vector or negative control. In still other embodiments, a therapeutically effective amount may raise the carbachol-induced airway hyper-reactivity in a patient by at least about 1-fold, 2-fold, 3-fold, 4-fold, 5-fold or more, as compared to administration of a vector or negative control.

As is well known in the art, therapeutically effective amounts for use in humans can also be determined from animal models. For example, a dose for humans can be formulated to achieve a circulating concentration that has been found to be effective in animals. A particularly useful animal model for multiple sclerosis is the EAE mouse model (Beeton, et al., PNAS, 98: 13942-13947 (2001); Reich, et al, Eur. J. Immunol., 35: 1 (2005); Lars Madsen, et al., Eur. J. Immunol. 35: 10 (2005). The dosage in humans can be adjusted by monitoring KCa3.1 channel inhibition and adjusting the dosage upwards or downwards, as described above.

Adjusting the dose to achieve maximal efficacy in humans based on the methods described above and other methods as are well-known in the art is well within the capabilities of the ordinarily skilled artisan. In the case of local administration, the systemic circulating concentration of administered compound will not be of particular importance. In such instances, the compound is administered so as to achieve a concentration at the local area effective to achieve the intended result.

Patient doses for oral administration of the compounds described herein, which is the preferred mode of administration for prophylaxis and for treatment of asthma or inflammatory process episodes, typically range from about 1 mg/day to about 1,000 mg/day, more typically from about 1 mg/day to about 100 mg/day, or from about 3 mg/day to about 70 mg/day, or from about 10 mg/day to about 50 mg/day, or from about 20 mg/day to about 40 mg/day, or from about 10 mg/day to about 40 mg/day, or from about 1 mg/day to about 10 mg/day. Stated in terms of patient body weight, typical dosages range from about 0.01 to about 50 mg/kg/day, more typically from about 0.1 to about 15 mg/kg/day, and most typically from about 0.1 to about 10 mg/kg/day. In certain embodiments, the dosage range may be from about 0.05 mg/kg/day to about 10 mg/kg/day, or from about 0.05 mg/kg/day to about 5 mg/kg/day, or about 0.05 mg/kg/day to about 2 mg/kg/day, or from about 0.05 mg/kg/day to about 1 mg/kg/day, or about 0.05 mg/kg/day to about 0.5 mg/kg/day.

In certain embodiments, a dosage for a compound of the invention may comprise an amount sufficient to maintain a baseline level of drug in the plasma of a patient. For example, a dosage sufficient to maintain at least about 500 ng/ml of the administered compound in the plasma of a patient. In other embodiments, the dosage may be sufficient to maintain at least about 250 ng/ml compound, or at least about 100 ng/ml, or at least about 75 ng/ml, 50 ng/ml, 40 ng/ml, 30 ng/ml, 25 ng/ml, 20 ng/ml, or 10 ng/ml of the administered compound in the blood of a patient. In certain embodiments, the dosage necessary to maintain a baseline amount of a compound of the invention in the plasma of an individual may comprise a range of about 1 to about 100 mg/day, or about 3 to about 70 mg/day, or about 10 to about 50 mg/day, or about 20 to about 40 mg/day.

Compounds of the invention that are useful in the methods of the invention have a half life in human blood or other mammalian blood which is >5 hours; but other useful compounds of the invention have a half life >10 hours; especially useful compounds have a half life in blood which are between 10 hours and 20 hours; and preferably have a half life which exceeds 15 hours or 20 hours or 25 hours in blood. Formulations which use a control-release formula to extend the amount of time between doses of the compound are also contemplated.

For other modes of administration, dosage amount and interval can be adjusted individually to provide plasma levels of the administered compound effective for the particular clinical indication being treated. For example, if acute inflammatory processes are the most dominant clinical manifestation, in one embodiment, a compound according to the invention can be administered in relatively high concentrations multiple times per day. Alternatively, if the patient exhibits only periodic inflammatory crises on an infrequent, periodic or irregular basis, in one embodiment, it may be more desirable to administer a compound of the invention at minimal effective concentrations and to use a less frequent administration regimen. This will provide a therapeutic regimen that is commensurate with the severity of the individual's inflammatory disease.

In certain embodiments, the compounds of the present invention may be administered once daily. In other embodiments, the compounds may be administered 2 or more times daily. In other embodiments, the compounds of the invention may be administered once every two days, or about once every 3, 4, 5, or 6 days. In yet other embodiments, the compounds of the invention may be administered once weekly, or about once or twice monthly. Given the long in vivo half lives of the some of the compounds of the invention, for example Senicapoc, it may be advantageous to dose less frequently than typical asthma related therapies. In certain embodiments, the compounds of the invention may be administered prophylactically. In other embodiments, the compounds of the invention may be administered therapeutically in response to an event, such as an asthma attack or exercise induced bronchial spasm.

Utilizing the teachings provided herein, an effective prophylactic or therapeutic treatment regimen can be planned which does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

In certain embodiments, the compounds of the invention used for formulation of drugs used to treat asthma or an inflammatory disease are substantially purified prior to formulation. In certain embodiments, the purity of the compounds is at least about 80%, preferably at least about 90%, or at least about 95%, 96%, 98%, 99%, 99.5%, or greater purity as measured by HPLC using RI detection, detection at 254 nm wavelength, or detection at 220 nm wavelength of substances present in the purified material.

### Compound Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED50 with little or no toxicity. The dosage may vary within this range depending upon the dosage form employed and the route of administration utilized. See, e.g., In The Pharmacological Basis of Therapeutics, Ch. 1, p. 1, 1975. The exact formulation, route of administration and dosage can be chosen by the individual physician in view of the patient's condition and the particular method in which the compound is used.

### Methods

In addition to the compounds and pharmaceutical formulations discussed in detail above, the present invention provides a number of methods in which the compounds of the invention find use. The methods range from those that might be used in a laboratory setting to probe the basic mechanisms of, for example, pharmacokinetics, drug activity, disease origin and progression and the like.

The invention is particularly useful in treating or preventing asthma and inflammatory diseases. An “inflammatory process” as used herein is a disease in which lymphoproliferation contributes to tissue or organ damage leading to disease, especially in cases where mast cell migration and mast cell degranulation with histamine release are also present. For instance, excessive T-cell proliferation at the site of a tissue or organ will cause damage to the tissue or organ, as will the presence of stimulated mast cells. Inflammatory processes are well known in the art and have been described extensively in medical textbooks (See, e.g., Harrison's Principles of Experimental Medicine, 13th Edition, McGraw-Hill, Inc., N.Y.).

In an exemplary embodiment, the present invention provides a method for treating or preventing an inflammatory process, involving administering to a subject suffering from an inflammatory process a therapeutically effective amount of a compound having the structure according to Formula I, II, III, IV or V.

Disease associated with abnormalities of the inflammatory process include but are not limited to proliferative glomerulonephritis; lupus erythematosus; scleroderma; temporal arteritis; thromboangiitis obliterans; mucocutaneous lymph node syndrome; host versus graft syndrome; inflammatory bowel disease; cancer; multiple sclerosis; rheumatoid arthritis; thyroiditis; Grave's disease; pulmonary eosinophilia; Guillain-Barre syndrome; allergic rhinitis; myasthenia gravis; human T-lymphotrophic virus type 1-associated myelopathy; herpes simplex encephalitis; inflammatory myopathies; atherosclerosis; Goodpasture's syndrome, insulin-dependent (Type 1) diabetes mellitus, peripheral neuritis, experimental autoimmune myocarditis and pulmonary hypertension. Some examples of asthma or inflammatory processes as well as animal models for testing and developing the compounds are set forth in Table 2 below.

TABLE 2

| Disease | Proliferating Cells | Reference | Animal Model | Reference |
|---|---|---|---|---|
| Asthma | T-cells | Hogg 1997 APMIS | Airway inflammation | Henderson et al. |
| | | 100: 105(10) | and hyper- | 1997 J Clin Invest |
| | | | Ovalbumin | 100(12) 3083-3092 |
| | | | sensitized mice or | |
| | | | guinea pigs. | |
| Glomeru- | Mesangial | Nitta et al. 1997 | NZB/NZW crossed | Clynes et al. 1998 |
| lonephritis | (glomerular) cells | Eur J. Pharmacol | mice develop | Science 279 |
| | | 344: 107-110 | glomerular disease | (5353): 1052-54. |
| | | | and lupus-like | |
| | | | syndrome. | |
| Host versus Graft | T-cells | Schorlemmer et al. | Renal allograft | Lazarivuts et al. |
| | B cells | 1997 Int J Tissue | rejection in mice. | 1996 Nature 380 |
| | | React 19: 157-61. | | (6576) 717-720. |
| | | J. Immunol | | |
| | | 160: 5320-30 | | |
| Inflammatory | Epithelial cells | Bajaj-Elliott et al. | Trinitro-benzene | Boughton-Smith et |
| Bowel Disease | | 1997 Am J. Pathol. | sulphonic acid | al. 1988 Br J |
| | | 151: 1469-76 | induced bowel | Pharmacol 94: 65-72. |
| | | | inflammation in rats. | |
| Systemic Lupus | Glomerular cells | Kodera et al. 1997 | NZB/NZW crossed | Peng et al. 1996 |
| Erythematosis | Lymphocytes | Am J Nephol | mice develop | Mol Biol Rep 23(3-4): |
| | | 17: 466-70. Akashi | glomerular disease | 247 51. |
| | | et al. 1998 | and lupus-like | |
| | | Immunology | syndrome. | |
| | | 93: 238-48 | | |
| Multiple | T-cells | Constantinesecu et | Experimental allergic | Drescher et al. 1998 |
| Sclerosis | | al. 1998 Immunol | encephalmyelitis. | J Clin Invest |
| | | Res 17(1-2): 217-27. | | 101(8): 1765-74. |
| Rheumatoid | T-cells Synovial | Ceponis et al. 1998 | Rat adjuvant arthritis | Anderson et al. |
| Arthritis | cells | Br J Rheumatol | assay | 1996 J Clin Invest |
| | | 37(2): 170-8 | | 97(11): 2672-9. |
| Thyroiditis | T-cells and | Rose et al. 1997 | HLA transgenic mice | J Clin Investig |
| | Epithelial cells | Crit Rev Immunol | immunized with | 101(5): 921-6. |
| | | 17: 511-7. | thryoglobulin. | |
| | | Schumm-Draeger | | |
| | | et al. 1996 Verh | | |
| | | Dtsch Ges Pathol | | |
| | | 80: 297-301. | | |
| Grave's Disease | Thyroid cells | DiPaola et al. 1997 | Thiouracil-fed rats. | Vilietto et al. 1997 |
| | | J Clin Endocrinol | | Oncogene 15: 2687 |
| | | Metab 82: 670-3. | | 98. |
| Antigen-induced | T-cells | Wolyniec et al. | | |
| airway | | 1998 Am J Respir | | |
| hyperactivity | | Cell Mol Biol | | |
| | | 18: 777-85 | | |
| Pulmonary | T-cells | Wolyniec et al. | | |
| eosinophilia | | 1998 Am J Respir | | |
| | | Cell Mol Biol | | |
| | | 18: 777-85 | | |
| Guillain-Barre | T-cells | Hartung et al. 1991 | Experimental | |
| Syndrome | | Ann Neurol. 30: 48-53 | autoimmune neuritis | |
| (inflammatory | | | (immunization with | |
| demyelinating | | | PNS myelin and | |
| disease) | | | Freunds complete | |
| | | | adjuvant) | |
| GianT-cell | T-cells | Brack et al. 1997 | | |
| arteritis (a form | | Mol Med 3: 530-43 | | |
| of systemic | | | | |
| vasculitis) | | | | |
| Inflammation of | | | | |
| large arteries. | | | | |
| Allergic Rhinitis | T-cells | Baraniuk et al. | | |
| | | 1997 J Allergy Clin | | |
| | | Immunol 99: S763-72 | | |
| Myasthenia | T-cells | Hartung et al. 1991 | | |
| gravis | | Ann Neurol 30: 48-53 | | |
| Human T- | T-cells | Nakamura et al. | | |
| lymphotropic | | 1996 Intern Mede | | |
| virus type 1- | | 35: 195-99 | | |
| associated | | | | |
| myelopathy | | | | |
| Herpes simplex | T cells | Hartung et al. 1991 | | |
| encephalitis | | Ann Neurol 30: 48-53 | | |
| Inflammatory | T-cells | Hartung et al. 1991 | | |
| myopathies (ie. | | Ann Neurol 30: 48 | | |
| Polymyositis, | | 53 Lindberg et al. | | |
| dermatomyocitis) | | 1995 Scan J | | |
| | | Immunol 141: 421-26 | | |
| Artherosclerosis | T-calls | Rosenfeld et al. | | |
| | | 1996 Diabetes Res | | |
| | | Clin Pract 30 | | |
| | | suppl.: 1-11 | | |
| Goodpasture's | Macrophages | Lan et al. 1995 Am | | |
| syndrome | | J Pathol 147: 1214-20 | | |

Thus, in one aspect, the invention provides a method for treating or preventing an inflammatory process, said method comprising administering to a subject suffering from said inflammatory process a therapeutically effective amount of a compound according to Formula (I), Formula (II), Formula (III), Formula (IV), or Formula (V) as set forth above.

Thus, in another aspect, the present invention provides for a method of treating or preventing asthma. The method includes administering to a subject suffering from asthma a therapeutically effective amount of a compound having a structure according to Formula (I), Formula (II), Formula (III), Formula (IV), or Formula (V). In another exemplary embodiment, the method involves treating asthma by administering a compound of the invention to a mammal not otherwise in need of treatment with the compounds of the invention.

In another embodiment, the compounds and formulations of the present invention may be administered simultaneously with other drugs or therapies known to treat asthma. Non-limiting examples of asthma treatments include bronchodilators or beta-2 agonist bronchodilators, such as albuterol, metaproterenol, pirbuterol, levalbuterol, theophylline, salmeterol, formoterol, advair, tiotropium, and ipratopium; corticosteroids, including inhaled corticosteroids and systemic corticosteroids, such as beclomethasone, triamcinolone, flunisolide, fluticasone, and budesonide; mast cell stabilizers, such as cromolyn; leukotriene blockers, such as zafirlukast, montelukast, and zileuton; and anti-IgE antibodies, such as omalizumab. In one embodiment, the invention provides a method of treating asthma, comprising administering a compound of Formula (I), Formula (II), Formula (III), Formula (IV), or Formula (V) to a patient in need thereof, simultaneously with a second anti-asthma treatment.

In an exemplary embodiment of the methods of the invention according to any of the aspects, the subject treated using the methods does not have sickle cell disease.

In summary, the invention provides methods for treating or preventing various disease states. Accordingly, the invention provides a method for treating or preventing asthma and/or an inflammatory process. The method includes administering to a subject suffering from asthma or the inflammatory process or at risk of suffering from asthma and/or an inflammatory process a therapeutically effective amount of a compound according to Formula I:

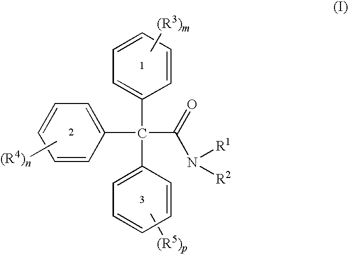

wherein R³, R⁴, and R⁵ are independently selected from F and CF₃, wherein m, n and p are independently selected from 0, 1, 2, and 3, wherein at least one of m, n and p is not 0, and wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl. In one embodiment, at least one of R¹ and R² is an alkyl selected from methyl, ethyl, n-propyl, isopropyl, n-butyl, and isobutyl. In a particular embodiment at least one of R¹ and R² is H. In another particular embodiment, both of R¹ and R² are H.

In a further set of embodiments, the methods of the invention include administering compounds of the invention wherein m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent. When p is 0, and m is 1 and n is 1, the fluoro substituent at ring 1 is para to the acetamide substituent, and the substituent at ring 2 is located at a position selected from ortho to the acetamide substituent and para to the acetamide substituent.

The present invention also provides a method for treating or preventing asthma or decreasing inflammation or nitric oxide in an asthmatic subject or in a subject at risk of developing asthma. The method includes administering to a subject suffering from asthma or at risk of developing asthma a therapeutically effective amount of a compound according to Formula (I), Formula (II), Formula (III), Formula (IV), or Formula (V).

The present invention also provides a method for treating or preventing or decreasing airway inflammation or respiratory nitric oxide production in an asthmatic subject or a subject at risk of developing asthma. The method includes administering to a subject suffering from asthma or at risk of developing asthma a therapeutically effective amount of a compound according to Formula (I), Formula (II), Formula (III), Formula (IV), or Formula (V). In a further embodiment, respiratory or pulmonary nitric oxide production is monitored to assess the efficacy of the treatment or response of the patient to therapy. The presence of nitric oxide, for instance, can be monitored in exhaled air.

Also provided is a method according to any of the paragraph above, wherein the compound has a structure according to Formula II:

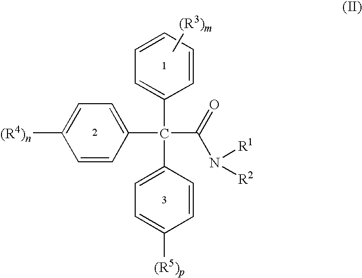

wherein R³, R⁴, and R⁵ are independently selected from F and CF₃, wherein m, n and p are independently selected from 0, 1, 2, and 3, wherein at least one of m, n and p is not 0, and wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl. In one embodiment, at least one of R¹ and R² is an alkyl selected from methyl, ethyl, n-propyl, isopropyl, n-butyl, and isobutyl. In a particular embodiment at least one of R¹ and R² is H. In another particular embodiment, both of R¹ and R² are H.

Another embodiment provides a method according to any of the paragraphs above, wherein the compound has a structure according to Formula III:

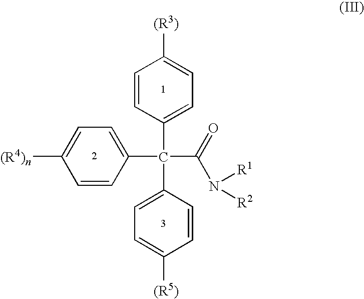

wherein R³, R⁴, and R⁵ are independently selected from F and CF₃, wherein n is 0, 1, 2, or 3, and wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl. In one embodiment, at least one of R¹ and R² is an alkyl selected from methyl, ethyl, n-propyl, isopropyl, n-butyl, and isobutyl. In a particular embodiment at least one of R¹ and R² is H. In another particular embodiment, both of R¹ and R² are H.

Another embodiment provides a method according to any of the paragraphs above, wherein the compound has a structure according to Formula IV:

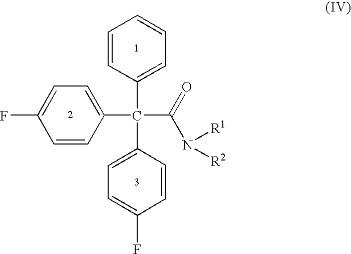

wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl. In one embodiment, at least one of R¹ and R² is an alkyl selected from methyl, ethyl, n-propyl, isopropyl, n-butyl, and isobutyl. In a particular embodiment at least one of R¹ and R² is H. In another particular embodiment, both of R¹ and R² are H.

Another embodiment of the invention provides a method according to any of the paragraphs above, wherein the compound has a structure according to Formula V:

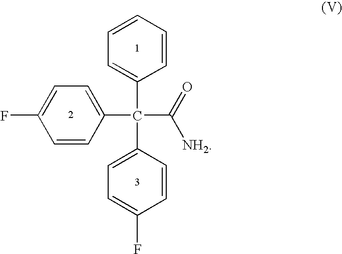

Also provided is a method according to any of the paragraphs above in which the compound has a structure that is selected from:

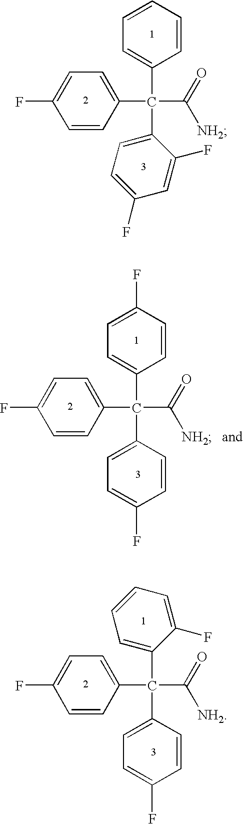

The invention also provides a method of any of the paragraphs above, wherein the disease state is mediated by a potassium channel.

In an exemplary method according to of any of the paragraphs above, the potassium channel is IK1 or KCa3.1.

The invention further provides a method for treating or preventing allergen-induced airway hyperreactivity and/or inhibiting mast cell migration, and/or release/degranulation. The method includes administering to a subject in need thereof a therapeutically effective amount of a compound according to Formula (I), Formula (II), Formula (III), Formula (IV), or Formula (V).

In some embodiments, the invention further provides a method for treating or preventing asthma, intermittent asthma, mild to moderate asthma, exercise-induced asthma or allergen-induced asthma. The method includes administering to a subject in need thereof a therapeutically effective amount of a compound according to Formula (I), Formula (II), Formula (III), Formula (IV), or Formula (V). In further embodiments, the compound stabilizes the mast cell or inhibits mast cell migration and/or release.

In some embodiments, the invention further provides a method for stabilizing mast cells or modulating mast cell or release, treating or preventing late airway reactivity in asthma, or treating or preventing airway hyper-reactivity in asthma by modulating or stabilizing mast cells or mast cell release. The method includes administering to a subject in need thereof a therapeutically effective amount of a compound according to Formula (I), Formula (II), Formula (III), Formula (IV), or Formula (V).

In preferred embodiments of any of the above aspects and aspects where the compound is to be administered, the compound is administered orally, by injection or by inhalation. In preferred embodiments, the compound is administered once or twice a day, or every other day, for a period of at least 1 week, 2 weeks or 1 month.

In an exemplary embodiment according to any of the paragraphs above, the subject treated using the method set forth in any of the paragraphs above does not have sickle cell disease.

The compounds, compositions and methods of the present invention are further illustrated by the examples that follow. These examples are offered to illustrate, but not to limit the claimed invention.

### EXAMPLES

Example 1 illustrates methods for the synthesis and characterization of compounds of the invention. The compounds of the invention were isolated in substantially pure form and in good yields utilizing the methods detailed in this Example. Other synthetic methods are disclosed in U.S. Pat. No. 6,288,122 and U.S. Pat. No. 6,028,103.

Example 2 describes the characterization of Senicapoc: Bis(4-fluorophenyl)phenyl acetamide, activity for the inhibition of the K⁺ channel protein KCa3.1.

Example 3 illustrates the attenuation of allergen-induced asthma in sheep by administration of Senicapoc.

Example 4 describes formulations of Senicapoc for therapeutic administration in sheep.

Example 5 describes a formulation of Senicapoc for therapeutic administration in humans.

Example 6 describes the effects of Senicapac on airway responsiveness to allergens.

### Example 1

This Example illustrates methods for the synthesis and characterization of compounds of the invention. The compounds of the invention were isolated in substantially pure form and in good yields utilizing the methods detailed below. The example provides methods of general scope that can be used to synthesize compounds of the invention other than those specifically exemplified.

### 1.1 Materials and Methods

Reagents were used as received unless otherwise stated. The method of Franco et al., J. Chem. Soc. Perkins Trans. II, 443 (1988), was used to prepare non-commercial fluorophenyllithium reagents and fluorobenzophenones. All moisture-sensitive reactions were performed under a nitrogen atmosphere using oven dried glassware. Reactions were monitored by TLC on silica gel 60 F₂₅₄ with detection by charring with Hancssian's stain (Khadem et al., Anal. Chem., 30: 1958 (1965)). Column chromatography was carried out using Selecto silica gel (32-63 μm). Melting points were determined on an Electrothermal IA9000 unit and are uncorrected. ¹H (300 MHz) and ¹⁹F (282 MHz) spectra were recorded on a Varian (Gemini 2000) NMR machine at room temperature in CDCl₃. Tetramethylsilane was used as the internal reference. Chiral separation of compound 1 was performed by Chiral Technologies using a CHLIRACEL1 toreq. OD-R column and acetonitrile/water as the eluant.

### 1.2 Preparation of Compound 1

Compound 1 was prepared in 28% yield in four steps from commercially available precursors.

### 1.2a Synthesis of (2-fluorophenyl)-(4-fluorophenyl)phenylmethanol

Phenylmagnesium bromide (1.83 mL, 5.5 mmol) was added dropwise to a stirring solution of 2,4′-difluorobenzophenone (1.09 g, 5.0 mmol) in t-butylmethyl ether (12 mL) at room temperature (“rt,” about 25° C.): After the addition was complete the reaction was heated at reflux for 3 h. The solution was cooled tort and was poured in to ice cold 1.0 M HCl (aq) (20 mQ. The organics were extracted with EtOAc (3×10 mL) and dried (Na₂SO₄). Concentration under reduced pressure gave the desired product (2-fluorophenyl)-(4-fluorophenyl)phenylmethanol as a pale brown oil which was used in the next reaction without any further purification.

### 1.2b Synthesis of (2-fluorophenyl)-(4-fluorophenyl)phenylacetonitrile

(2-Fluorophenyl)-(4-fluorophenyl)phenylmethanol (1.47 g, 5.0 mmol) was added to a 20% solution of acetyl chloride in dichloromethane (10 mL) at rt. The resulting solution was stirred for 12 h after which the solvent was removed by evaporation. Toluene (2×20 mL) was added to the residue and evaporated to afford crude 2-fluorophenyl-(4-fluorophenyl)phenylchloromethane which was used without purification in the next step.

Copper cyanide (0.50 g, 5.5 mmol) was added to the residue and the resultant mixture was heated at 130° C. for 2.5 h. Once the reaction had cooled to approximately 110° C. toluene (30 mL) was added and the mixture was stirred vigorously for 10 min. The mixture was filtered and the solvent was removed under reduced pressure. Hot hexane (30 mL) was added to the crude material and the mixture was stirred vigorously for 30 min. Filtration and washing with more hexane gave the desired cyan product as a white solid, which was used without further purification.

### 1.2c Synthesis of (2-fluorophenyl)-(4-fluorophenyl)phenylacetamide (1)

A solution of concentrated sulfuric acid (10 mL) and glacial acetic acid (10 mL) was added to crude (2-fluorophenyl)-(4-fluorophenyl)phenylacetonitrile (1.48 g, 5.0 mmol) at rt. The resulting orange solution was stirred and heated at 130° C. for 3 h. The reaction was cooled to 0° C. and was neutralized by the dropwise addition of ammonium hydroxide. Water was added (30 mL) and the organics were extracted with chloroform (3×30 mL). The organic fractions were combined and washed sequentially with water (2×10 mL) and brine (20 mL). The organic phase was dried (Na₂SO₄) and concentrated under reduced pressure. Hexane (30 mL) was added to the resulting light brown oil to initiate precipitation. The precipitate was ground up and washed sequentially with hot hexane (30 mL). Crystallization from hexane/dichloromethane gave the desired product (2-fluorophenyl)-(4 fluorophenyl)phenylacetamide as a white crystalline solid (0.45 g, 1.4 mmol, 28%, 4 steps).

### 1.3 Preparation of Compound 3

Compound 3 was prepared in three steps from commercially available precursors in 58% yield.

### 1.3a Synthesis of bis(4-fluorophenyl)phenylmethanol

Phenylmagnesium bromide (100 mL, 0.1 mol) was added dropwise to a stirring solution of 4,4′-difluorobenzophenone (20 g, 0.092 mol) in t-butylmethyl ether (150 mL) at rt. After the addition was complete the reaction was heated at reflux for 3 h. The solution was cooled to rt and was poured in to ice cold aqueous 1.0 M HCl (100 mL). The organics were extracted with EtOAc (2×50 mL) and dried (Na₂SO₄). Concentration under reduced pressure gave bis(4-fluorophenyl)phenylmethanol as a pale brown oil. After drying in vacuo for 2 h the crude material was used in the next reaction without any further purification.

### 1.3b Synthesis of bis(4-fluorophenyl)phenylacetonitrile

Bis(4-fluorophenyl)phenylmethanol (0.092 mol) was added to a 20% solution of acetyl chloride in dichloromethane (50 mL) at rt. The resulting purple solution was stirred for 12 h after which the solvent was removed by evaporation. Toluene (100 mL) was added to the residue and then evaporated, affording crude bis(4-fluorophenyl)phenylchloromethane which was used without purification in the following step.

Copper cyanide (8.24 g, 0.11 mot) was added to the crude residue and the mixture was heated at 140° C. for 3 h. The reaction was cooled to 100° C. and toluene (100 mL) was added. The resulting mixture was stirred vigorously for 10 min, cooled to rt, filtered through a short pad of silica and the solvent was removed under reduced pressure to afford a brown solid. Hot hexane (100 mL) was added to the powdered crude material and the mixture was stirred vigorously for 4 h. Filtration and washing with additional hexane gave the desired bis(4-fluorophenyl)phenylacetonitrile as a white solid (18.9 g, 67%).

### 1.3c Synthesis of bis(4-fluorophenyl)phenylacetamide (3)

A solution of concentrated sulfuric acid (50 mL) and glacial acetic acid (50 mL) was added to bis(4-fluorophenyl)phenylacetonitrile (18.9 g, 0.06 mot) at rt. The resulting orange solution was stirred and heated at 130° C. for 3 h. The reaction was cooled to 0° C., poured into ice water (150 mL) and neutralized with ammonium hydroxide. The organics were extracted with chloroform (3×100 mL), combined and washed with brine (2×50 mL). The organics were dried (Na₂SO₄) and concentrated under reduced pressure to afford a yellow-orange solid. The solid was stirred with hot hexane (100 ml) for 30 min and filtered. Crystallization from dichloromethane/hexane gave bis(4-fluorophenyl)phenylacetamide (3) as a white crystalline solid (16.9 g, 0.052 mot, 87%).

### 1.4 Preparation of Compound 5

Compound 5 was prepared in 66% yield in four steps from commercially available precursors.

### 1.4a Synthesis of bis(4-fluorophenyl)-2-fluorophenylmethanol

p-Fluorophenylmagnesium bromide (124 mL, 0.12 mot) was added dropwise to a stirring solution of 2,4′-difluorobenzophenone (24.5 g, 0.11 mol) in t-butylmethyl ether (100 mL) at rt. After the addition was complete the reaction was heated at reflux for 3 h. The solution was then cooled tort and was poured in to ice cold 1.0 M HCl (aq) (100 mL). The organics were extracted with EtOAc (3×70 mL) and dried (Na₂SO₄). Concentration under reduced pressure gave the desired product bis(4-fluorophenyl)-2-fluorophenylmethanol as a pale yellow oil which was used in the next reaction without any further purification.

### 1.4b Synthesis of bis(4-fluorophenyl)-2-fluorophenylacetonitrile

A 20% solution of acetyl chloride in dichloromethane (60 mL) was added to the crude bis(4-fluorophenyl)-2-fluorophenylmethanol at rt. The resulting solution was stirred for 12 h after which the solvent was removed by evaporation. Toluene (100 mL) was added to the residue and was then evaporated to afford crude bis(4-fluorophenyl)-2-fluorophenyl chloromethane which was used without purification in the next step.

Copper cyanide (12 g, 0.13 mol) was added to the crude material and the resulting mixture was heated at 160° C. for 3 h. The reaction was cooled to approximately 110° C., toluene (100 mL) was added and the mixture was stirred vigorously for 10 min. The mixture was cooled, filtered through a short silica plug and concentrated under reduced pressure. Hot hexane (100 mL) was added to the crude material and the mixture was stirred vigorously for 30 min. Filtration and washing with more hexane gave the desired bis(4-fluorophenyl)-2-fluorophenylacetonitrile as a white solid (25.3 g, 70%).

### 1.4c Synthesis of bis(4-fluorophenyl)-2-fluorophenylacetamide (5)

A solution of concentrated sulfuric acid (10 mL) and glacial acetic acid (10 mL) was added to bis(4-fluorophenyl)-2-fluorophenylacetonitrile (5.0 g, 0.015 mol) at rt. The resulting orange solution was stirred and heated at 130° C. for 2 h. The reaction was cooled to 0° C. and was poured onto ice (50 g). The resulting mixture was neutralized by the dropwise addition of ammonium hydroxide. Methylene chloride (100 mL) was added and the organics were extracted with additional methylene chloride (3×30 mL). The combined organic fractions were washed sequentially with water (2×10 mL) and brine (20 mL). The organic phase was dried (Na₂SO₄) and concentrated under reduced pressure to afford a yellow/orange solid. The solid was powdered and washed repeatedly with hot hexane (50 ml) until no coloration was evident in the filtrate. Crystallization from hexane/dichloromethane gave the desired product bis(4-fluorophenyl)-2-fluorophenylacetamide 5 as a white crystalline solid (4.98 g, 0.0145 mol, 94%).

### 1.5 Preparation of Compound 16

Compound 16 was prepared in 11% yield in four steps from commercially available precursors.

### 1.5a Synthesis of bis(4-fluorophenyl)-3-fluorophenylmethanol

n-Butyllithium (4 mL, 10 mmol) was added dropwise to a stirring solution of bromo-3-fluorobenzene (1.75 g; 10 mmol) in THF (25 mL) at −78° C. After 20 min 4,4′-benzophenone (1.96 g, 9 mmol) was added. The reaction was allowed to warm to 0° C. over a 30 min period. Saturated ammonium chloride (aq) (30 mL) was added and stirring was continued for 30 min. EtOAc (20 mL) was added, the organics were separated, washed with brine (20 mL), dried (Na₂SO₄) and concentrated under reduced pressure. The residue was purified by column chromatography (100% hexane to 100% methylene chloride) to afford bis(4-fluorophenyl)-3-fluorophenylmethanol (2.81 g, 92%).

### 1.5b Synthesis of bis(4-fluorophenyl)-3-fluorophenylacetonitrile

Bis(4-fluorophenyl)-3-fluorophenylmethanol (999 mg, 3.18 mmol) was added to a 20% solution of acetyl chloride in dichloromethane (10 mL) at rt. The resulting purple solution was stirred for 12 h after which the solvent was removed by evaporation. Toluene (20 mL) was added to the residue and then evaporated affording crude bis(4-fluorophenyl)-3-fluorophenylchloromethane which was used in the next step without purification.

Copper cyanide (344 mg, 3.82 mmol) was added to the crude material and the resulting mixture was heated at 140° C. for 3 h. The reaction was cooled to approximately 110° C., toluene (50 mL) was added and the mixture was stirred vigorously for 10 min. The mixture was cooled to rt, filtered through a short pad of silica and the solvent. was removed under reduced pressure to afford a beige solid. Hot hexane (100 mL) was added to the powdered crude material and the mixture was stirred vigorously for 1 h. Filtration and washing with additional hexane gave bis(4-fluorophenyl)-3-fluorophenylacetonitrile as a white solid which was used without further purification.

### 1.5c Synthesis of bis(4-fluorophenyl)-3-fluorophenylacetamide (16)

A solution of concentrated sulfuric acid (10 mL) and glacial acetic acid (10 mL) was added to bis(4-fluorophenyl)-3-fluorophenylacetonitrile (3.18 mmol) at rt. The resulting orange solution was stirred and heated at 130° C. for 3 h. The reaction was cooled to 0° C., poured into ice water (50 mL) and neutralized with ammonium hydroxide. The organics were extracted with chloroform (3×50 mL). The organics fractions were combined washed with brine (2×20 mL), dried (Na₂SO₄) and concentrated under reduced pressure to afford a yellow-orange solid. The solid was stirred with hot hexane (50 ml) for 30 min and filtered.

Crystallization from dichloromethane/hexane gave the desired product bis(4-fluorophenyl)-3-fluorophenylacetamide 16 as a white crystalline solid (147 mg, 0.43 mmol, 11%, 4 steps).

### 1.6 Compound Characterization by ¹H and ¹⁹F NMR Spectroscopy and Melting Point

The compounds of the invention were characterized by a combination of ¹H and ¹⁹F NMR spectroscopy and the compound melting points were determined.

1: ¹H NMR δ (CDCl₃): 7.39-7.26 (8H, m), 7.15-6.90 (5H, m), 5.83 (1H, brs), 5.72 (1H, brs); ¹⁹F NMR δ (CDCl₃): −103.4 (1F, s), −115.8 (1F, s); m.p 180-181° C.

2: ¹H NMR δ (CDCl₃): 7.37-7.28 (6H, m), 7.15-7.05 (2H, m), 6.93 (1H, dt, J=8 and 2 Hz), 5.90 (1H, brs), 5.68 (1H, brs); ¹⁹F NMR δ (CDCl₃): −103.4 (1F, m); m.p 210° C.

3: ¹H NMR δ (CDCl₃): 7.37-7.20 (9H, m), 7:04-6.91 (4H, m), 5.81 (1H, brs), 5.71 (1H, brs); ¹⁹F NMR δ (CDCl₃): −115.7 (2F, s); m.p 180-181° C.

4: ¹H NMR δ (CDCl₃): 7.37-7.24 (12H, m), 6.97 (2H, t, J=8.5 Hz), 5.83 (1H, brs), 5.75 (1H, brs); ¹⁹F NMR δ (CDCl₃): −116.2 (1F, s); m.p 193-194° C.

5: ¹H NMR δ (CDCl₃): 7.41-7.34 (1H, m), 7.29-7.23 (4H, m), 7.16 (1H, ddd, J=18.1, 8.1 and 1.2 Hz), 7.15 (1H, d, J=7.7 Hz), 7.05-6.97 (4H, m), 6.93-6.87 (1H, dt, J=8.0 and 1.4 Hz), 5.90 (1H, brs), 5.74 (1H, brs); ¹⁹F NMR δ (CDCl₃): −103.3 (1F, s), −115.5 (2F, s); m.p 168-169° C.

6: ¹H NMR δ (CDCl₃): 7.64-7.54 (4H, m), 7.40-7.34 (6H, m), 5.70 (2H, brs); ¹⁹F NMR δ (CDCl₃): 137.3 (2F, d, J=19.2 Hz), −155.8 (1F, t, J=21.4 Hz), −161.9 (2F, dd, J=21.4 and 17.1 Hz).

7: ¹H NMR δ (CDCl₃): 7.37-7.31 (6H, m), 7.28-7.20 (5H, m), 7.12-7.04 (2H, m), 5.90 ((1H, brs), 5.74 (1H, brs). ¹⁹F NMR δ (CDCl₃): −137.8 to −137.9 (1F, m), −140.3 to −140.4 (1F, m); m.p 174-175° C.

8: ¹HNMR δ (CDCl₃): 7.37-7.28 (10H, m), 6.95-6.83 (2H, m), 6.81-6.75 (1H, m), 5.92 (111, brs), 5.80 (H, brs); ¹⁹F NMR δ (CDCl₃): −99.1 (1F, dd, J=19.2 and 8.5 Hz), −111.6 (1F, m); m.p 187-188° C.

9: ¹H NMR δ (CDCl₃): 7.38-7.22 (7H, m), 7.09-6.96 (6H, m), 5.83 (1H, brs), 5.77 (1H, brs); ¹⁹F NMR δ (CDCl₃): −112.6 (2F, dd, J=17.1 and 6.4 Hz); m.p 195-196° C.

13: ¹H NMR δ (CDCl₃): 7.26-7.19 (6H, dd, J=9.0 and 5.4 Hz), 7.20-7.01 (6H, t, J=8.7 Hz), 5.83 (1H, brs), 5.69 (1H, brs); ¹⁹F NMR δ (CDCl₃): −115.3 (3F, s); m.p 180-181° C.

14: ¹H NMR δ (CDCl₃): 7.39-7.27 (9H, m), 7.17-7.03 (4H, m), 5.90 (1H, brs), 5.85 (1H, brs). ¹⁹F NMR δ (CDCl₃): −102.9 (2F, s); m.p 166-167° C.

15: ¹H NMR δ (CDCl₃): 7.41-7.34 (2H, m), 7.29-7.23 (4H, m), 7.17-7.05 (4H, m), 6.99 (2H, t, J=8.7 Hz), 5.78 (2H, brs); ¹⁹F NMR δ (CDCl₃): −103.0 (2F, s), −115.9 (1F, m); m.p 187-188° C.

16: ¹H NMR δ (CDCl₃): 7.34-7.20 (6H, m), 7.06-6.97 (6H, m), 5.90 (1H, brs), 5.71 (1H, brs); ¹⁹F NMR δ (CDCl₃): −112.2 (1F, dd, J=17.1 and 7.4 Hz), −115.1 to −115.2 (2F, m); m.p 165-166° C.

17: ¹H NMR δ (CDCl₃): 7.35-7.21 (3H, m), 7.06-6.97 (9H, m), 7.17-7.05 (4H, m), 5.96 (1H, brs), 5.76 (1H, brs); ¹⁹F NMR δ (CDCl₃): −112.2 (3F, dd, J=17.1 and 8.5 Hz); m.p 186-188° C.

### Example 2

The inhibitory activity of Senicapoc: Bis(4-fluorophenyl)phenyl acetamide, on the K⁺ channel protein KCa3.1 was characterized in studies involving CHO cells stably expressing KCa3.1 and isolated human mast cells.

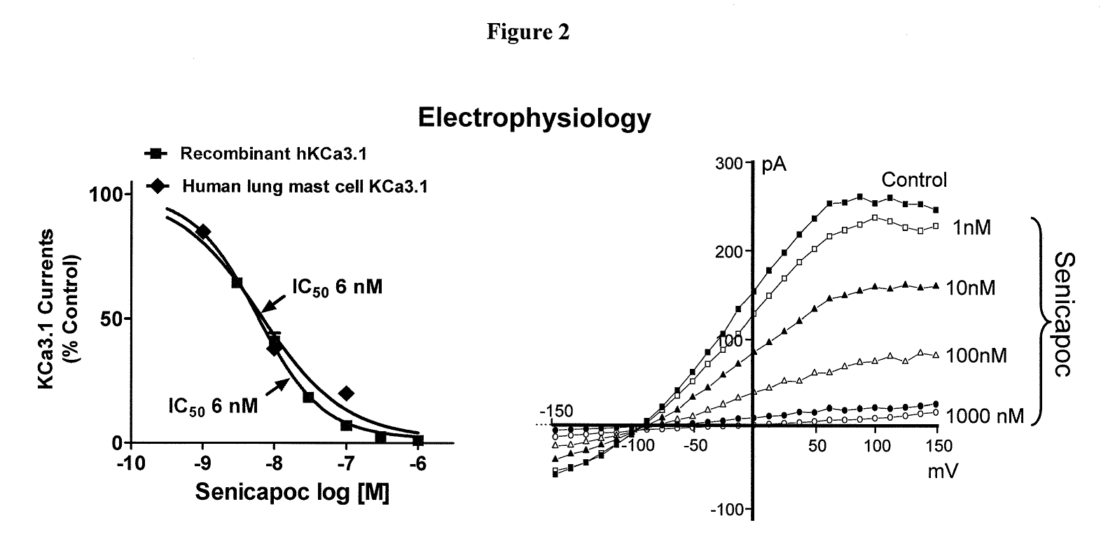

Potassium currents were recorded using whole patch clamp techniques in CHO cells stably expressing recombinant human KCa3.1 or isolated human lung mast cells (FIG. 2). KCa3.1 currents in CHO cells were stimulated by elevating intracellular Ca2⁺ to 1 μM whereas KCa3.1 currents in mast cells were activated by application of the known opener EBIO (100 μM). As can be seen in the left panel of FIG. 2, Senicapoc inhibited KCa3.1 K⁺ channels expressed on the surface of both recombinant CHO cells and isolated human mast cells to a similar extent (IC₅₀˜6 nM). The voltage applied in the patch clamp experiments was then varied and current across the cellular membrane measured in the presence of 0, 1, 10, 100, and 1,000 nM Senicapoc. As seen in the right panel of FIG. 2, nearly complete channel inhibition was obtained in the presence of 1000 nM Senicapoc.

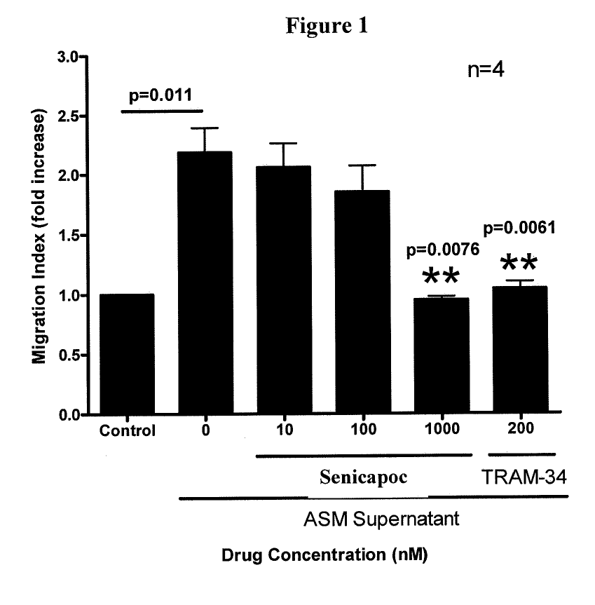

Mast cell migration studies were then performed by a method similar to as described in the method of Cruse et al. (Thorax. 2006 61:880-5). Briefly, HLMC chemotaxis assays were performed using the Transwell system with 24 wells. Airway smooth muscle-conditioned media was placed in the lower wells. Senicapoc (50 μl of 2× the final concentration of senicapoc with the final being 10, 100, and 1000 nM) or a positive control (TRAM-34) was added to the upper chambers before the addition of 1×10⁵ HLMC per well (50 μl). After incubating the cells for 3 hours at 37° C., the number of HLMC in the bottom well were counted using Kimura stain in a hemocytometer. HLMC migration was calculated as the fold increase of migrated cells in the test wells compared with the negative control containing no chemoattractant in the lower well (FIG. 1).

As seen in the experiments described above, Senicapoc inhibits endogenous KCa3.1 potassium currents in human lung mast cells as well as migration resulting from inflammatory stimuli.

### Example 3

The ability of Senicapoc to attenuate allergen-induced asthma was studied using the in vivo Ascaris suum allergen challenge model of asthma developed in sheep. The present example demonstrates that Senicapoc is able to attenuate allergen-induced asthma in sheep when administered intravenously, orally, or via inhalation.

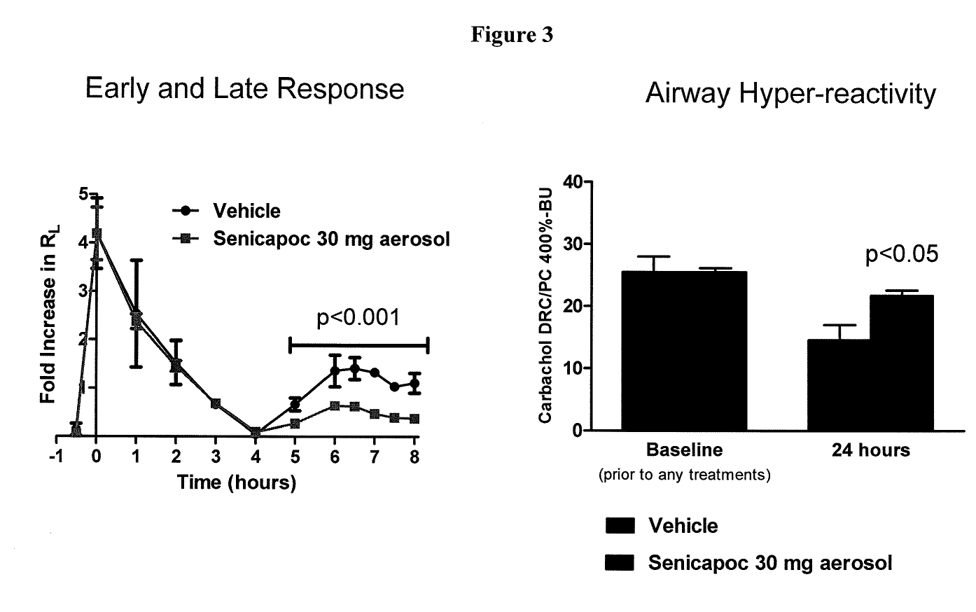

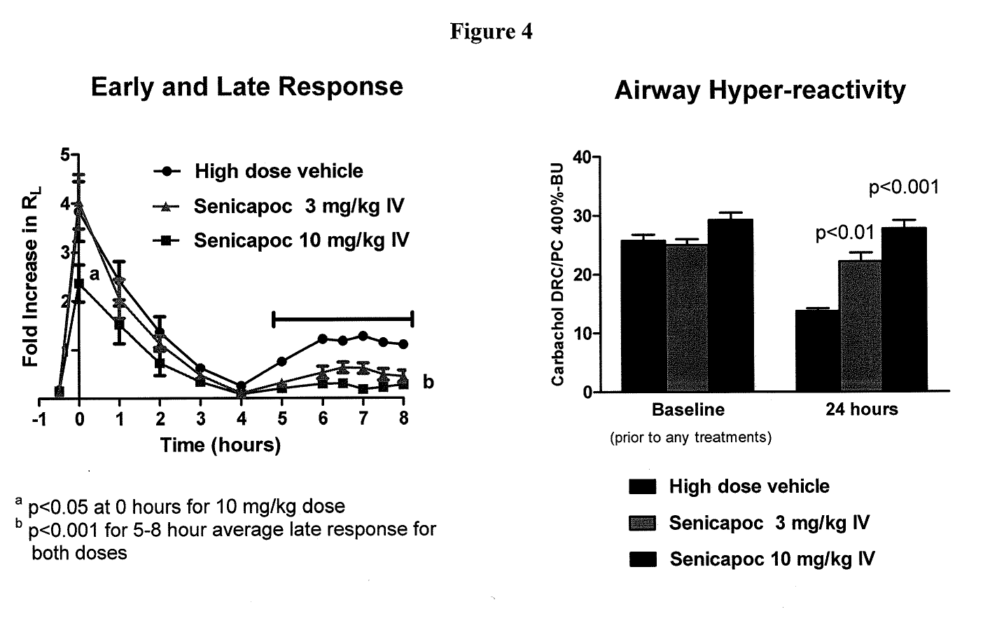

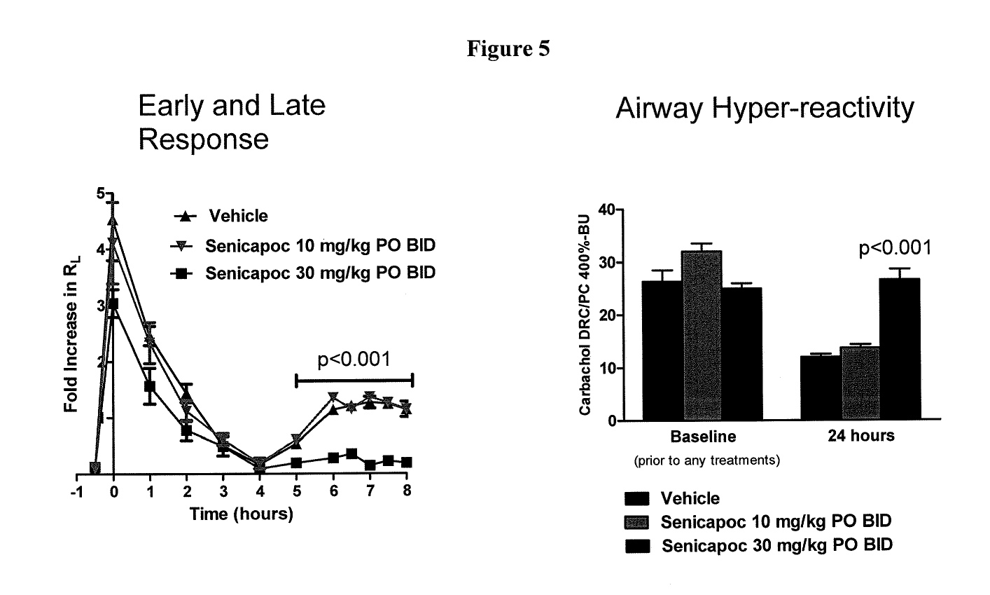

To test the efficacy of Senicapoc in the treatment of asthma, the sheep asthma model was performed as described in Abraham (Pulm Pharmacol Ther. 2008 21:743-54). Briefly, baseline lung resistance (RL) and carbachol concentration response curves were obtained prior to A. suum challenge in sheep. The sheep were then administered Senicapoc either via inhalation (in aerosol formulation) (FIG. 3), intravenously (FIG. 4), or orally (FIG. 5). For PO (oral) dosing, Senicapoc was administered b.i.d. at 30 mg/kg or 10 mg/kg 4 days prior to challenge with dosing continuing throughout the testing period. When given intravenously, Senicapoc was administered once, 15 minutes before A. suum challenge at 10 mg/kg (vehicle of NMP:PEG 400:water; 2:5:3) or 3 mg/kg (vehicle of NMP:PEG400:water; 2:3:5). For aerosol delivery, Senicapoc (30 mg total dose) was dissolved in ethanol and administered with a disposable medical nebulizer, the same system used for delivering A. suum and carbachol.

Lung resistance was subsequently measured at 0, 1, 2, 3, and 4 hours post allergen challenge to assess early airway responses and at 5, 6, 6.5, 7, 7.5 and 8 hours to assess late airway responses. At 24 hours post challenge, airway hyper-reactivity was determined based on responses to increasing doses of carbachol. Data were analyzed by a one way ANOVA followed by Dunnett's post hoc test when more than 2 groups were compared, or by a t-test when only 2 groups were compared.

As can be seen in FIGS. 3 to 5, Senicapoc significantly attenuates average late allergen responses (5-8 hours after allergen challenge) in A. suum sensitized sheep, regardless of the route of administration. Significant effects were observed by the oral, IV and inhalation routes of administration. Peak early airway responses (observed immediately after allergen challenge) were significantly reduced by Senicapoc dosed at 10 mg/kg IV. There was a 33% apparent reduction in peak early response at the 30 mg/kg PO dose, although this effect was not statistically significant. Hyper-reactivity to aerosolized carbachol at 24 hours post A. suum challenge was significantly reduced by senicapoc by all three routes of administration. Complete reversal of the hyper-reactivity was achieved by dosing 30 mg/kg PO for 4 days and by acute 10 mg/kg IV dosing.

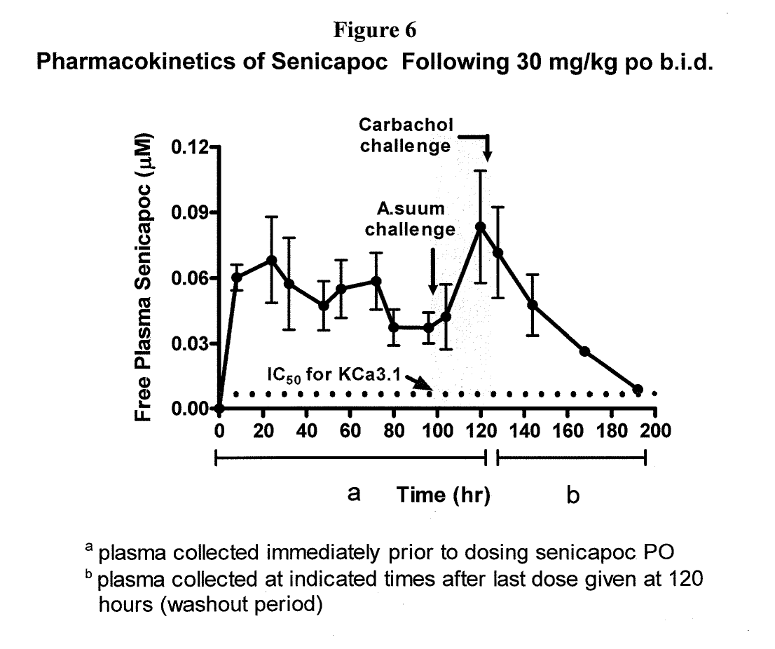

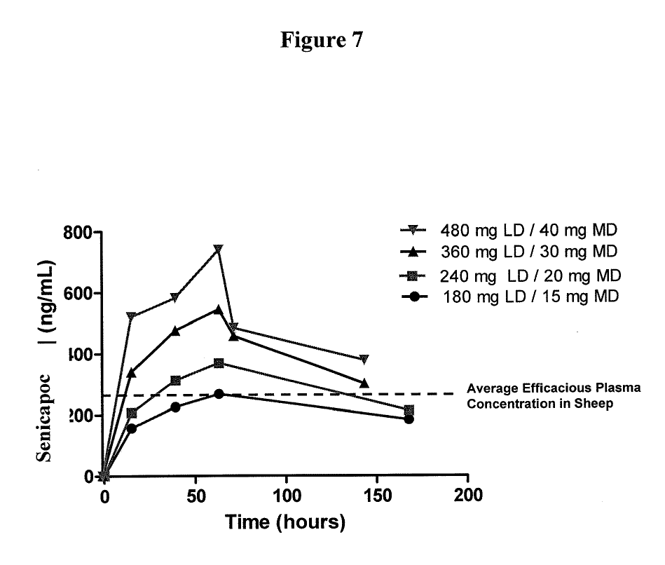

As can be seen in FIG. 6, the plasma exposure corrected for protein binding in the sheep model achieves sustained levels significantly above those required to block KCa3.1 (30 mg/kg, po b.i.d). Since statistically significant effects were observed on lung resistance and airway hyper-reactivity following the 30 mg/kg dose, this level of exposure is sufficient to achieve efficacy in the sheep model. FIG. 7 illustrates exposure in man following administration of a range of doses. The average efficacious plasma level from the sheep model is illustrated by the dashed line. Exposure levels above that required for efficacy in the sheep model are achieved at all time points measured following maintenance doses of 30 mg and 40 mg in man.

### Example 4

The formulation of Senicapoc for administration to sheep, as in Example 3 varied depending on the route of administration. In general, for administration by inhalation to sheep, a compound of interest was dissolved in ethanol and was administered via a nebulizer for inhaled administration. Oral administration in sheep was performed using a formulation of a compound of interest in a methylcellulose vehicle. For i.v. use in sheep, the compound of interest was dissolved in a vehicle comprising NMP:PEG 400:water; 2:5:3 for a dose of 10 mg/kg and in a vehicle comprising NMP:PEG400:water; 2:3:5 for a 3 mg/kg dose, wherein NMP means N-methylpyrrolidone and PEG-400 means polyethyleneglycol-400.

### Example 5

For oral administration in humans, 10 mg swallowable tablets, as well as 2 mg chewable tablets were formulated comprising the compound of interest, Senicapoc. Briefly, Sodium Lauryl Sulfate (0.246 kg; NF Grade) was dissolved in USP water (40.8 kg; purified) to make a granulating solution. In a High Shear Mixer Granulator the following materials are combined:
a. Lactose Monohydrate (58.14 kg; NF)
b. Ferric Oxide Yellow, (0.204 kg; NF)
c. Pregelatinized Starch, (11.22 kg; NF)
d. An active substance, such as Senicapoc (4.08 kg)
e. Microcrystalline Cellulose, (18.12 kg; NF)
f. Povidone K29/32, (5.10 kg; USP)
g. Croscarmellose Sodium, (4.08 kg; NF).

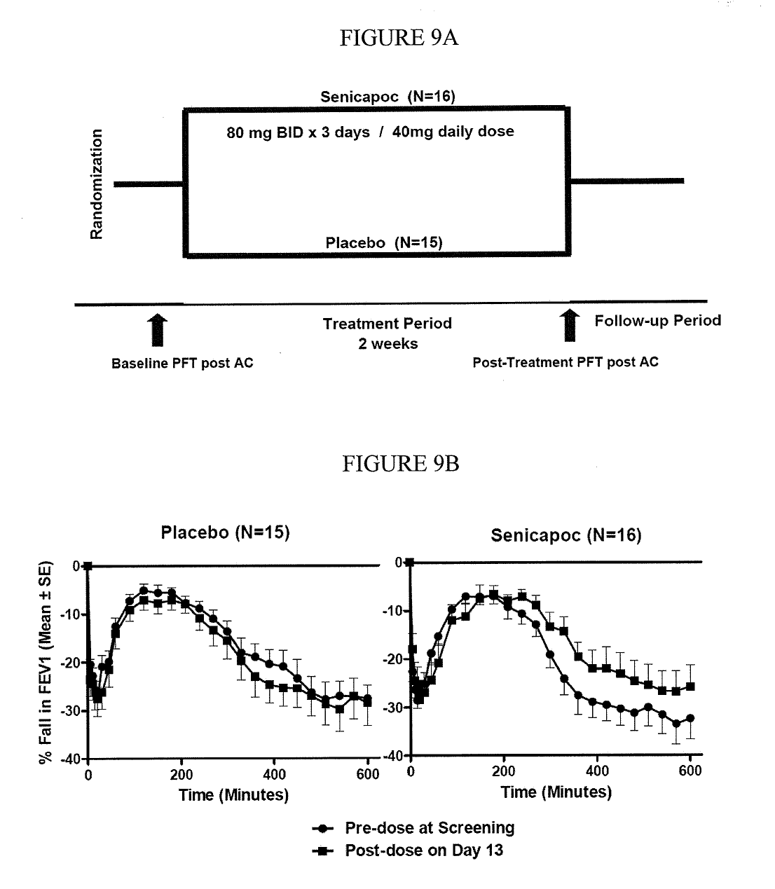

The dry ingredients were then dry-blended and granulated using the granulating solution above. The granulated mixture was then wet-milled, and the wet granules then dried. The dried granules were then milled, and then are blended with magnesium stearate (0.819 kg; NF) which had been previously screened. The lubricated blend is then compressed on a tablet press at a compression weight sufficient to deliver 10 mg of Senicapoc. The tablets are then packaged in the appropriate container/closure system. The dry blending, wet granulation, wet milling, drying, and milling of dry granules can be performed in portions if all portions are blended with the magnesium stearate lubricant to form one uniform batch for compressing into tablets. The processing procedure is diagramed in FIG. 9.

### Example 6

Subjects were randomly assigned to placebo or Senicapoc treatment groups (see, FIG. 9A). Predose screening showed no difference between the two groups in late airway responsiveness to a challenge allergen as monitored for 10 hours. After 13 days of treatment with placebo or Senicapoc, the responsiveness of the placebo and Senicapoc-treated subjects diverged. By analysis of co-variance, the difference in late airway responsiveness between the two treatment groups was 29% (see, FIG. 9B). In addition, the fraction of nitric oxide exhaled by Senicapoc-treated group declined with no change seen in the placebo group (data not shown).

It is understood that the examples and embodiments described herein are for illustrative purposes only and that various modifications or changes in light thereof will be suggested to persons skilled in the art and are to be considered included within the spirit and purview of this application and are considered within the scope of the appended claims. All publications, patents, and patent applications cited herein are hereby incorporated by reference in their entirety for all purposes.

What is claimed is:

1. A method for treating or preventing asthma, said method comprising administering to a subject suffering from asthma a therapeutically effective amount of a compound according to Formula I: 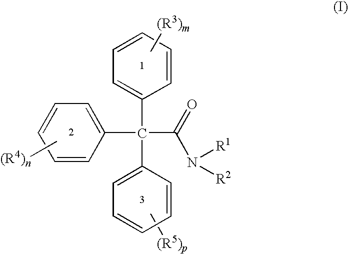
wherein,
(a) R³, R⁴, and R⁵ are independently selected from F and CF₃;
(b) m, n and p are independently selected from 0, 1, 2, and 3;
(c) at least one of m, n and p is not 0; and
(d) R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl.

2. The method according to claim 1, wherein said compound has a structure according to Formula II: 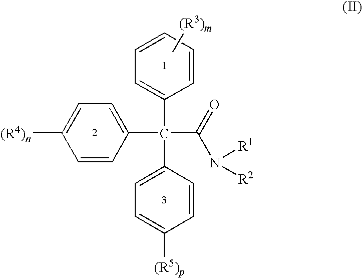
wherein,
(a) R³, R⁴, and R⁵ are independently selected from F and CF₃;
(b) m, n and p are independently selected from 0, 1, 2, and 3;
(c) at least one of m, n and p is not 0; and
(d) R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl.

3. The method according to claim 2, wherein said compound has a structure according to Formula III: 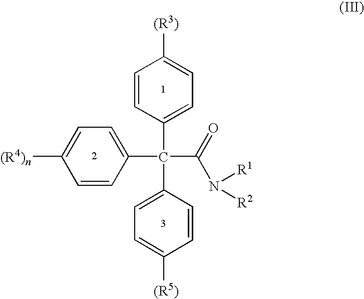
wherein,
(a) R³, R⁴, and R⁵ are independently selected from F and CF₃;
(b) n is selected from 0 and 1; and
(c) R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl.

4. The method of claim 3, wherein R¹ and R² are both H.

5. The method according to claim 1, wherein said compound has a structure according to Formula IV: 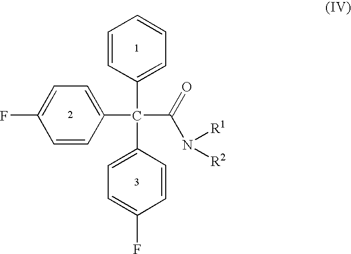
wherein R¹ and R² are independently selected from the group consisting of H, an alkyl, a substituted alkyl, an alkenyl, a substituted alkenyl, an aryl, a substituted aryl, a heteroaryl, a substituted heteroaryl, a heterocyclyl, a substituted heterocyclyl, an alkyl-O-alkyl, an alkyl-O-alkenyl, and a hydroxyl.

6. The method of claim 5, wherein at least one of R¹ and R² is H.

7. The method according to claim 6, wherein said compound has a structure according to Formula V:

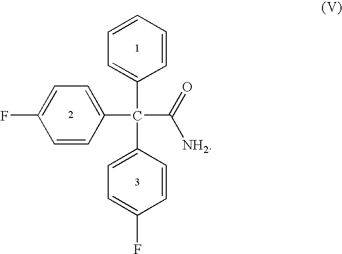

8. The method according to claim 1, said compound having a structure that is selected from:

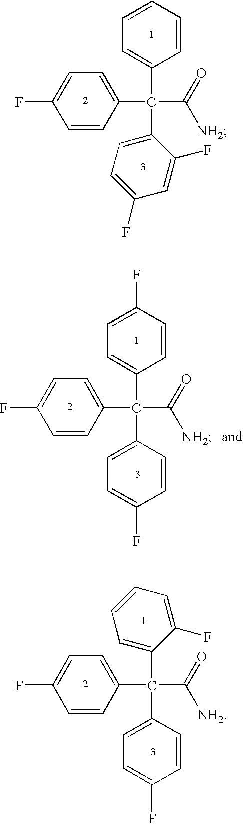

9. The method of claim 1, wherein said method further comprises administration of a second anti-asthma treatment.

10. The method of claim 1, wherein said compound is administered at from about 1 mg/day to about 100 mg/day.

11. The method of claim 10, wherein said compound is administered at from about 10 mg/day to about 40 mg/day.

12. The method of claim 11, wherein said dose is a maintenance dose.

13. The method of claim 1, wherein said compound is administered intravenously, orally, nasally, or via inhalation.

14. The method of claim 1, wherein the asthma is exercise-induced asthma or allergen-induced asthma.
